(19) United States
(12) Patent Application Publication
(10) Pub. No.: US 2020/0199261 A1
(43) Pub. Date: Jun. 25, 2020

(54) HIGHLY SENSITIVE OXIME ESTER PHOTOPOLYMERIZATION INITIATOR AND PHOTOPOLYMERIZABLE COMPOSITION INCLUDING THE SAME

(71) Applicants: Korea Research Institute of Chemical Technology, Daejeon (KR); TAKOMA TECHNOLOGY CO., LTD., Nonsan-si (KR)

(72) Inventors: Chang Jin LEE, Daejeon (KR); Jae Min LEE, Daejeon (KR); Shahid AMEEN, Daejeon (KR); Song Yun CHO, Daejeon (KR); Sung Cheol YOON, Daejeon (KR); Young Cheul LEE, Gumi-si (KR); Mi Sun RYU, Daejeon (KR); Bok Joo SONG, Daejeon (KR); Keun Soo KIM, Daejeon (KR); So Youn NAM, Daejeon (KR)

(73) Assignees: Korea Research Institute of Chemical Technology, Daejeon (KR); TAKOMA TECHNOLOGY CO., LTD., Nonsan-si (KR)

(21) Appl. No.: 16/316,085

(22) PCT Filed: Jul. 5, 2017
(86) PCT No.: PCT/KR2017/007132

§ 371 Date: Jan. 8, 2019

(30) Foreign Application Priority Data
Jul. 8, 2016 (KR) 10-2016-0087074

(51) Int. Cl. C08F 2/46; C08J 5/18; C07D 209/88; G03F 7/031; G03F 7/00; G02F 1/1335; G02F 1/1339; G02F 1/1333

(52) U.S. Cl. CPC C08F 2/46; C08J 5/18; C07D 209/88; G03F 7/031; G03F 7/0007; G02F 1/133514; G02F 2001/13398; G02F 1/1339; G02F 1/133305; C08J 2335/02; C08J 2435/02; C08J 2367/00; G02F 1/133512

(57) ABSTRACT

Provided is an oxime ester phenylcarbazole compound useful as a photoinitiator for photocrosslinking. Specifically, the carbon atom forming a double bond with the nitrogen atom in the oxime ester moiety of the oxime ester phenylcarbazole compound is bonded to the phenylcarbazole group and is directly bonded to a (C₁-C₂₀)alkyl or (C₆-C₂₀)aryl group. Also provided is a photopolymerizable composition including the oxime ester phenylcarbazole compound. The oxime ester phenylcarbazole compound and the photopolymerizable composition have improved solubilities, are highly photosensitive, and exhibit excellent physical properties in terms of residual film ratio, pattern stability, resist adhesiveness. Due to these advantages, the oxime ester phenylcarbazole compound and the photopolymerizable composition are suitable for use in black resists, color resists, overcoats, column spacers, and organic insulating films of LCDs.

### BACKGROUND OF THE INVENTION

### 1. Field of the Invention

The present invention relates to a highly sensitive oxime ester photopolymerization initiator and a photopolymerizable composition including the same.

### 2. Description of the Related Art

A photosensitive composition is prepared by adding a photopolymerization initiator to a polymerizable compound having an ethylenically unsaturated bond. Since the photosensitive composition can be cured by polymerization of the polymerizable compound when irradiated with polychromatic light at 365 nm, 405 nm, and 436 nm, it is used for photocurable inks, photosensitive printing plates, photoresists, etc. In order for the photosensitive composition to have sensitivity to short-wavelength light sources for attractive printing, the photopolymerization initiator is also required to have high sensitivity.

Many photopolymerization initiators for use in photosensitive compositions are known. For example, PCT International Publication No. WO 02/100903A1 describes photoinitiators having an oxime ester moiety. This patent publication discloses specific structures and synthesis methods of various oxime ester compounds that can be used as photoinitiators. However, in the case where such oxime ester compounds known in the art are used as photopolymerization initiators, their photodecomposition products are attached to masks, causing pattern defects during printing and leading to low yield.

The known photopolymerization initiators are decomposed during heat curing after development due to their low decomposition temperatures (≤240° C.). This decomposition deteriorates the adhesiveness of photosensitive compositions. For these reasons, there is a need for a highly sensitive photopolymerization initiator that is excellent in physical properties, such as pattern stability and adhesiveness.

### SUMMARY OF THE INVENTION

Therefore, one object of the present invention is to provide a highly sensitive photopolymerization initiator including an oxime ester moiety that produces no decomposition products attached to a mask when exposed to light and is excellent in adhesiveness, pattern stability, and sensitivity.

A further object of the present invention is to provide a photoinitiator compound that is moderately soluble in a solvent used in a photopolymerizable composition, and a photosensitive compound including the photoinitiator compound.

Still another object of the present invention is to provide a photosensitive composition including the oxime ester compound as a photoinitiator, particularly for a resist for the formation of a black matrix, a color filter, a black column matrix or a column spacer pattern, an organic insulating film or an overcoat with improved thin film characteristics.

One aspect of the present invention provides an oxime ester phenylcarbazole compound as a photoinitiator for use in photocrosslinking wherein the carbon atom forming a double bond with the nitrogen atom in the oxime ester moiety is bonded to the phenylcarbazole group and is directly bonded to a (C₁-C₂₀)alkyl or (C₆-C₂₀)aryl group and wherein the phenylcarbazole group is substituted with one or more nitro groups.

The phenylcarbazole group may be substituted with one or two nitro groups.

A further aspect of the present invention provides an oxime ester phenylcarbazole compound represented by Formula 1:

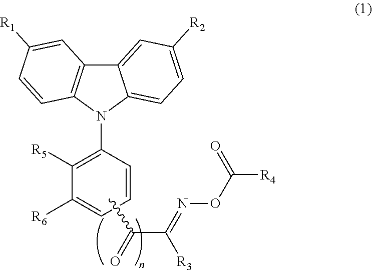

wherein R₁ and R₂ are each independently hydrogen, nitro, cyano, alkoxy or halogen, with the proviso that R₁ and R₂ are not simultaneously hydrogen, R₃ is (C₁-C₂₀)alkyl, (C₆-C₂₀)aryl, (C₁-C₂₀)alkoxy, (C₆-C₂₀)aryl(C₁-C₂₀)alkyl, hydroxy(C₁-C₂₀)alkyl, hydroxy(C₁-C₂₀)alkoxy(C₁-C₂₀)alkyl, (C₃-C₂₀)cycloalkyl, (C₁-C₂₀)alkylacyl or (C₆-C₂₀)arylacyl, R₄ is (C₁-C₂₀)alkyl, (C₆-C₂₀)aryl, (C₁-C₂₀)alkoxy, (C₆-C₂₀)aryl(C₁-C₂₀)alkyl, hydroxy(C₁-C₂₀)alkyl, hydroxy(C₁-C₂₀)alkoxy(C₁-C₂₀)alkyl, (C₃-C₂₀)cycloalkyl, (C₁-C₂₀)alkylacyl or (C₆-C₂₀)arylacyl; R₅ and R₆ are each independently hydrogen, (C₁-C₂₀)alkyl, (C₁-C₂₀)alkoxy, hydroxy(C₁-C₂₀)alkyl, hydroxy(C₁-C₂₀)alkoxy(C₁-C₂₀)alkyl, (C₃-C₂₀)cycloalkyl or (C₁-C₂₀)alkylacyl, and n is an integer of 0 or 1.

In Formula 1, R₁ and R₂ may be each independently hydrogen, nitro, cyano, alkoxy or halogen, with the proviso that one of R₁ and R₂ is nitro.

In Formula 1, R₃ may be (C₃-C₇)alkyl or (C₆-C₇)aryl.

In Formula 1, R₄ may be (C₁-C₃)alkyl or (C₆-C₅)aryl.

In Formula 1, R₅ and R₆ may be each independently hydrogen or (C₁-C₂)alkyl.

Another aspect of the present invention provides a photopolymerization initiator including the oxime ester phenylcarbazole compound.

Another aspect of the present invention provides a photopolymerizable composition including the oxime ester phenylcarbazole compound and at least one compound selected from polymeric compounds soluble in a solvent or aqueous alkaline solution and photopolymerizable compounds having an ethylenically unsaturated bond.

Another aspect of the present invention provides a photopolymerizable composition including the oxime ester phenylcarbazole compound, at least one compound selected from polymeric compounds soluble in a solvent or an aqueous alkaline solution and photopolymerizable compounds having an ethylenically unsaturated bond, and a dye or pigment.

Another aspect of the present invention provides a column spacer, a black matrix, a black column spacer, a color filter or a substrate having an organic insulating film formed from the photopolymerizable composition.

Yet another aspect of the present invention provides a substrate having a film formed by coating with the photopolymerizable composition.

The film may be a display panel of a TFT-LCD, OLED or TSP.

The oxime ester phenylcarbazole compound of the present invention is highly soluble in a solvent (e.g., PGMEA) used in a photosensitive composition. The use of the oxime ester phenylcarbazole compound as a photoinitiator for photocrosslinking can be minimized due to its high solubility. In addition, when the solvent is removed from a thin film of the photosensitive composition by evaporation, phase separation between a binder and the photoinitiator can be reduced, achieving improved characteristics of the thin film after crosslinking. Thus, the photosensitive composition can be used to provide a black matrix, a color filter, a column spacer, an insulating film or a photocrosslinkable coated substrate with good quality.

The oxime ester phenylcarbazole compound of the present invention is highly sensitive and has excellent physical properties in terms of residual film ratio, pattern stability, chemical resistance, and flexibility even when used in a small amount as a photopolymerization initiator of the photopolymerizable composition. Due to its excellent characteristics, the use of the oxime ester phenylcarbazole compound as a photopolymerization initiator can minimize the occurrence of outgassing during exposure and post-baking for the production of a resist for a display such as a TFT-LCD, OLED or TSP. This decreases the possibility of contamination, thus minimizing the number of defects.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 graphically compares the sensitivities of oxime ester phenylcarbazole compounds prepared in Examples 1, 2, and 3 and Comparative Examples 1, 2, 3, and 4 as photopolymerization initiators of photopolymerizable compositions.

### DETAILED DESCRIPTION OF THE INVENTION

The present invention will now be described in more detail.

The present invention provides an oxime ester phenylcarbazole compound useful as an initiator for the photopolymerization of a polymerizable compound having an ethylenically unsaturated bond due to its moderate solubility in a solvent and high sensitivity. The present invention also provides a photosensitive compound including the oxime ester phenylcarbazole compound.

The present invention also provides a photopolymerizable composition including the oxime ester phenylcarbazole compound as a photoinitiator for photocrosslinking.

The oxime ester phenylcarbazole compound as a photoinitiator for photocrosslinking has a structure in which the carbon atom forming a double bond with the nitrogen atom in the oxime ester moiety is bonded to the phenylcarbazole group and is directly bonded to a (C₁-C₂₀)alkyl or (C₆-C₂₀)aryl group.

The phenylcarbazole group is preferably substituted with one or more nitro groups. Specifically, the substitution of the phenylcarbazole group with one nitro group is effective in improving the sensitivity and solubility of the oxime ester phenylcarbazole compound. When the phenylcarbazole group is substituted with two or more nitro groups, high sensitivity is expected but low solubility is caused, making it difficult to use the oxime ester phenylcarbazole compound as a photoinitiator.

Specifically, the oxime ester phenylcarbazole compound as a photoinitiator for use in photocrosslinking may be represented by Formula 1:

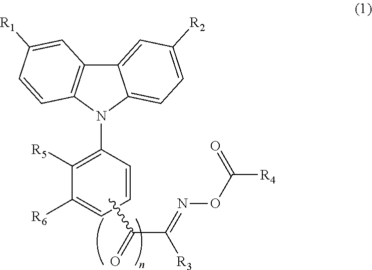

wherein R₁ and R₂ are each independently hydrogen, nitro, cyano, alkoxy or halogen, with the proviso that R₁ and R₂ are not simultaneously hydrogen, R₃ is (C₁-C₂₀)alkyl, (C₆-C₂₀)aryl, (C₁-C₂₀)alkoxy, (C₆-C₂₀)aryl(C₁-C₂)alkyl, hydroxy(C₁-C₂₀)alkyl, hydroxy(C₁-C₂₀)alkoxy(C₁-C₂₀)alkyl, (C₃-C₂₀)cycloalkyl, (C₁-C₂₀)alkylacyl or (C₆-C₂₀)arylacyl, R₄ is (C₁-C₂₀)alkyl, (C₆—C₂₀)aryl, (C₁-C₂₀)alkoxy, (C₆-C₂₀)aryl(C₁-C₂₀)alkyl, hydroxy(C₁-C₂₀)alkyl, hydroxy(C₁-C₂₀)alkoxy(C₁-C₂₀)alkyl, (C₃-C₂₀)cycloalkyl, (C₁-C₂₀)alkylacyl or (C₆-C₂₀)arylacyl; R₅ and R₆ are each independently hydrogen, (C₁-C₂₀)alkyl, (C₁-C₂₀)alkoxy, hydroxy(C₁-C₂₀)alkyl, hydroxy(C₁-C₂₀)alkoxy(C₁-C₂₀)alkyl, (C₃-C₂₀)cycloalkyl or (C₁-C₂₀)alkylacyl, and n is an integer of 0 or 1.

For reference, the alkyl group in the definition of the substituents consists of 1 to 20 carbon atoms unless otherwise mentioned herein.

R₁ and R₂ in Formula 1 may be each independently hydrogen, nitro, cyano, alkoxy or halogen. When at least one of R₁ and R₂ is nitro, the oxime ester phenylcarbazole compound is preferred in terms of sensitivity. The halogen may be F, Br or C₁.

R₃ in Formula 1 may be (C₃-C₇)alkyl or (C₆-C₇)aryl. When R₃ is (C₃-C₇)alkyl, the oxime ester phenylcarbazole compound is preferred in terms of solubility.

R₄ in Formula may be, for example, C₁-C₃ alkyl or C₆-C₈ aryl.

Specifically, R₄ may be methyl or phenyl.

R₅ and R₆ in Formula 1 may be each independently hydrogen or (C₁-C₂)alkyl.

Preferably, R₅ is hydrogen and R₆ is hydrogen or methyl. In this case, the oxime ester phenylcarbazole compound is imparted with high sensitivity.

When R₆ is hydrogen, the oxime ester moiety is in the para position relative to the carbazole group. When R₆ is methyl, R₆ and the oxime ester moiety are in the ortho and para positions relative to the carbazole group. The para position of the oxime ester moiety relative to the carbazole group is more preferred when steric hindrance is taken into account.

For example, n in Formula 1 is 0 or 1. When n is 1, the oxime ester phenylcarbazole compound exhibits higher sensitivity at longer wavelengths.

Specifically, the oxime ester phenylcarbazole compound as a photoinitiator for photocrosslinking or the oxime ester phenylcarbazole compound represented by Formula 1 is preferably selected from the group consisting of the following compounds:

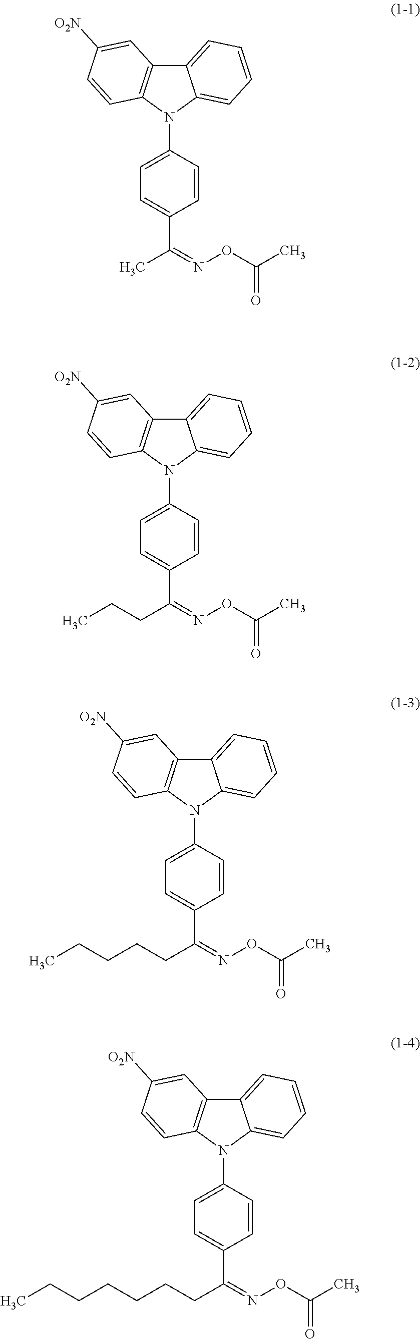

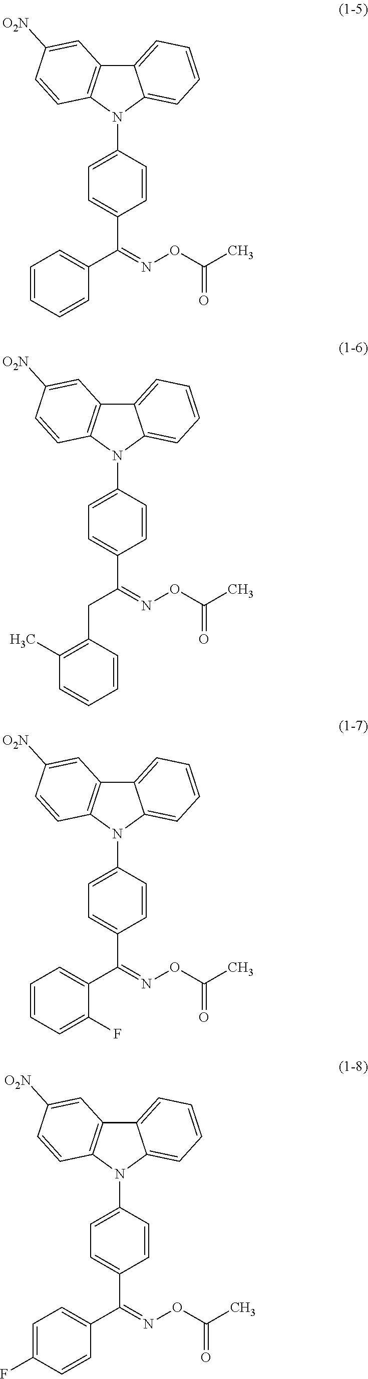 [SMILES: CC(=O)O/N=C(/c1ccccc1)c1ccc(-n2c3ccccc3c3cc([N+](=O)[O-])ccc32)cc1 ; CC(=O)O/N=C(/Cc1ccccc1C)c1ccc(-n2c3ccccc3c3cc([N+](=O)[O-])ccc32)cc1 ; CC(=O)O/N=C(\c1ccc(-n2c3ccccc3c3cc([N+](=O)[O-])ccc32)cc1)c1ccccc1F ; CC(=O)O/N=C(/c1ccc(F)cc1)c1ccc(-n2c3ccccc3c3cc([N+](=O)[O-])ccc32)cc1]

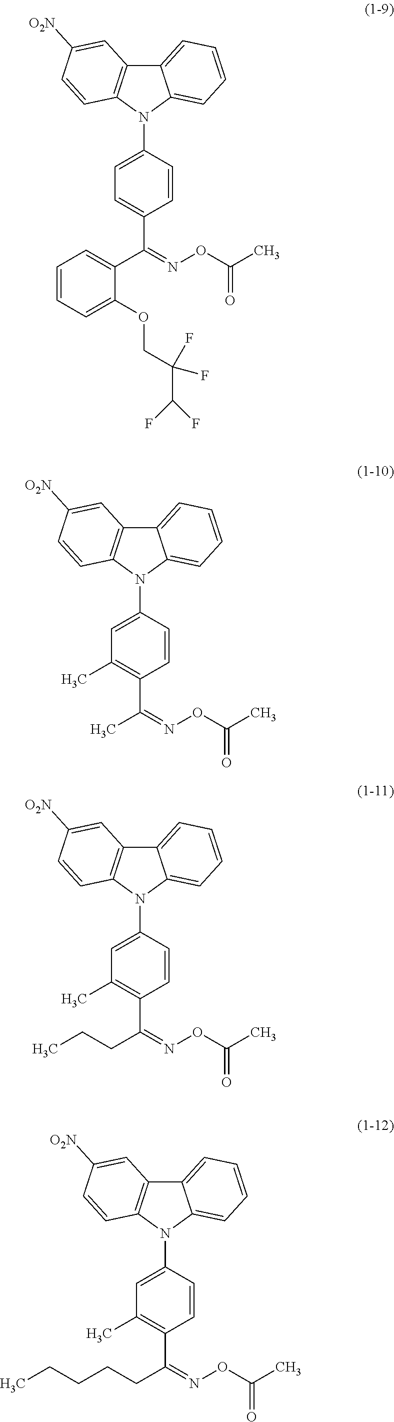

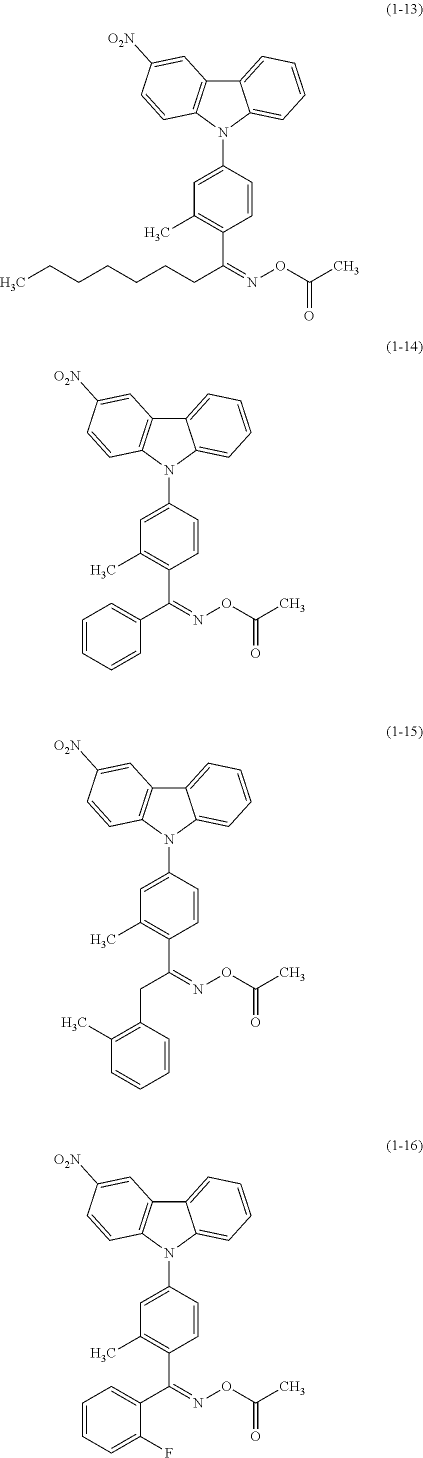 [SMILES: CCCCCCC/C(=N/OC(C)=O)c1ccc(-n2c3ccccc3c3cc([N+](=O)[O-])ccc32)cc1C ; CC(=O)O/N=C(/c1ccccc1)c1ccc(-n2c3ccccc3c3cc([N+](=O)[O-])ccc32)cc1C ; CC(=O)O/N=C(/Cc1ccccc1C)c1ccc(-n2c3ccccc3c3cc([N+](=O)[O-])ccc32)cc1C ; CC(=O)O/N=C(\c1ccc(-n2c3ccccc3c3cc([N+](=O)[O-])ccc32)cc1C)c1ccccc1F]

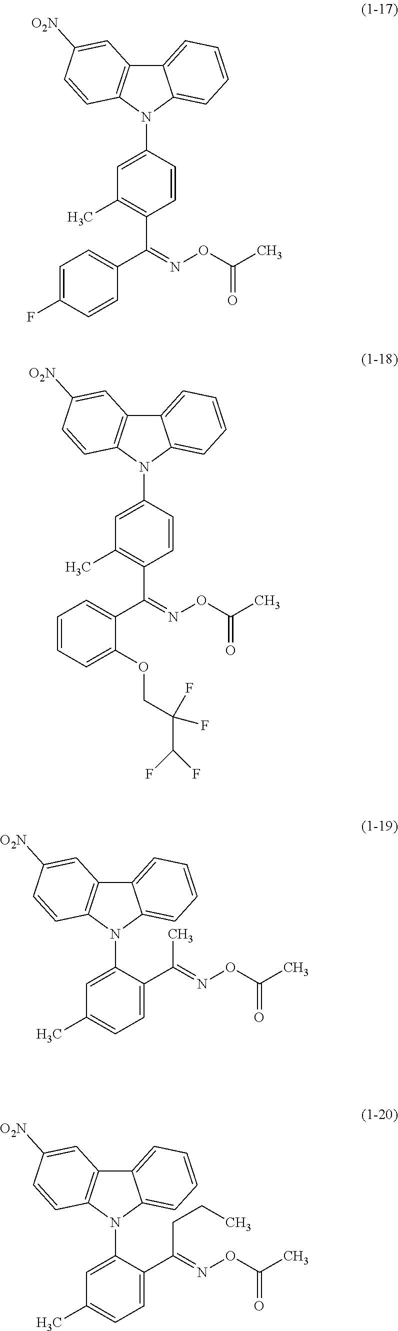 [SMILES: CC(=O)O/N=C(/c1ccc(F)cc1)c1ccc(-n2c3ccccc3c3cc([N+](=O)[O-])ccc32)cc1C ; CC(=O)O/N=C(\c1ccc(-n2c3ccccc3c3cc([N+](=O)[O-])ccc32)cc1C)c1ccccc1OCC(F)(F)C(F)F ; CC(=O)O/N=C(\C)c1ccc(C)cc1-n1c2ccccc2c2cc([N+](=O)[O-])ccc21 ; CCC/C(=N\OC(C)=O)c1ccc(C)cc1-n1c2ccccc2c2cc([N+](=O)[O-])ccc21]

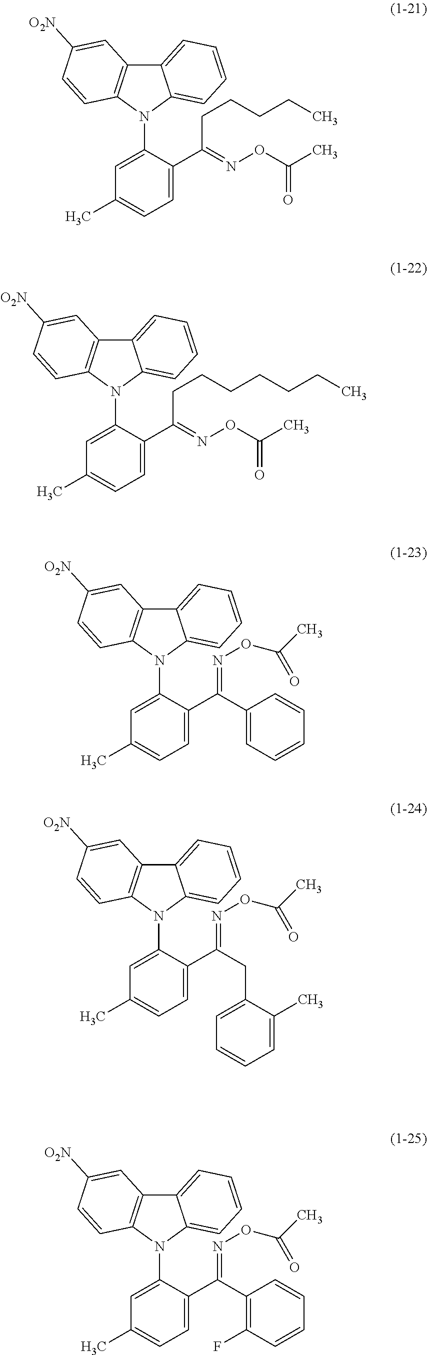 [SMILES: CCCCC/C(=N\OC(C)=O)c1ccc(C)cc1-n1c2ccccc2c2cc([N+](=O)[O-])ccc21 ; CCCCCCC/C(=N\OC(C)=O)c1ccc(C)cc1-n1c2ccccc2c2cc([N+](=O)[O-])ccc21 ; CC(=O)O/N=C(\c1ccccc1)c1ccc(C)cc1-n1c2ccccc2c2cc([N+](=O)[O-])ccc21 ; CC(=O)O/N=C(\Cc1ccccc1C)c1ccc(C)cc1-n1c2ccccc2c2cc([N+](=O)[O-])ccc21 ; CC(=O)O/N=C(\c1ccccc1F)c1ccc(C)cc1-n1c2ccccc2c2cc([N+](=O)[O-])ccc21]

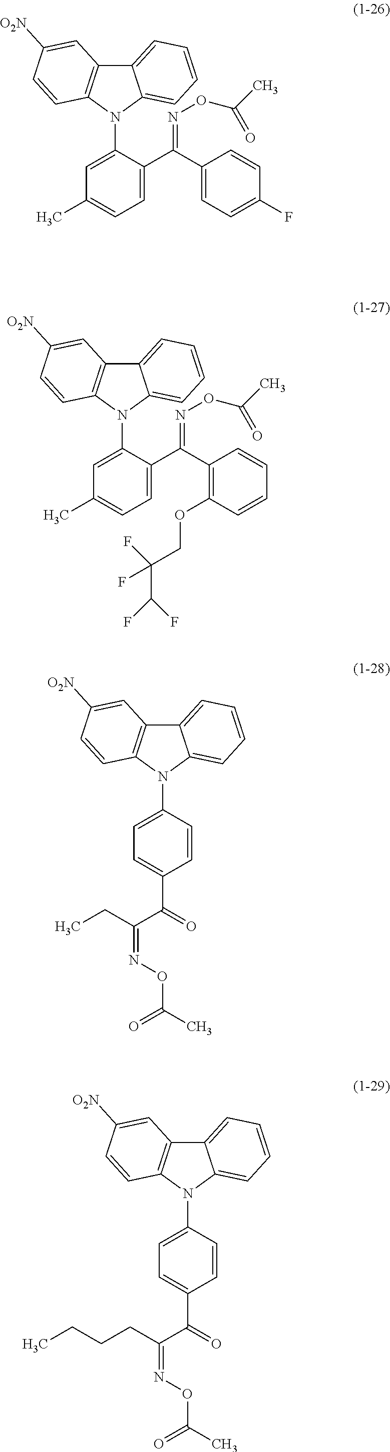 [SMILES: CC(=O)O/N=C(\c1ccc(F)cc1)c1ccc(C)cc1-n1c2ccccc2c2cc([N+](=O)[O-])ccc21 ; CC(=O)O/N=C(\c1ccccc1OCC(F)(F)C(F)F)c1ccc(C)cc1-n1c2ccccc2c2cc([N+](=O)[O-])ccc21 ; CC/C(=N/OC(C)=O)C(=O)c1ccc(-n2c3ccccc3c3cc([N+](=O)[O-])ccc32)cc1 ; CCCC/C(=N/OC(C)=O)C(=O)c1ccc(-n2c3ccccc3c3cc([N+](=O)[O-])ccc32)cc1]

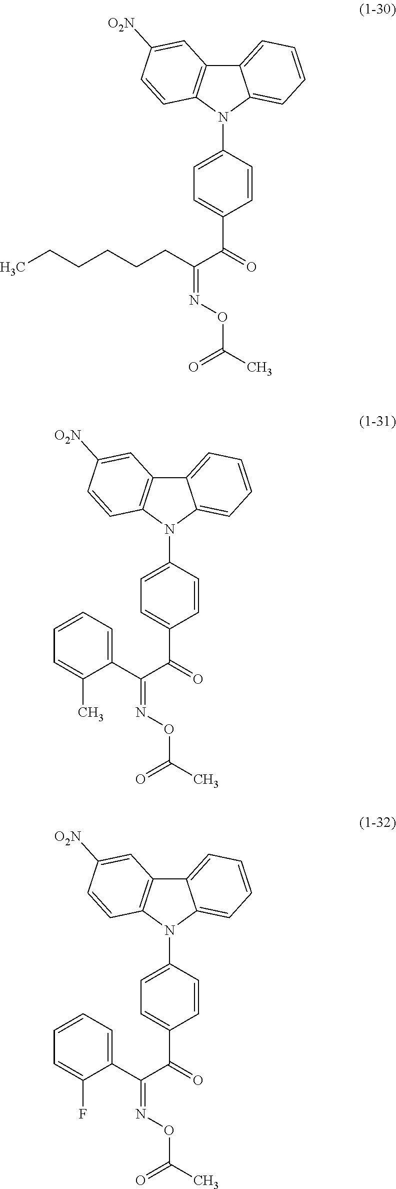 [SMILES: CCCCCC/C(=N/OC(C)=O)C(=O)c1ccc(-n2c3ccccc3c3cc([N+](=O)[O-])ccc32)cc1 ; CC(=O)O/N=C(\C(=O)c1ccc(-n2c3ccccc3c3cc([N+](=O)[O-])ccc32)cc1)c1ccccc1C ; CC(=O)O/N=C(\C(=O)c1ccc(-n2c3ccccc3c3cc([N+](=O)[O-])ccc32)cc1)c1ccccc1F]

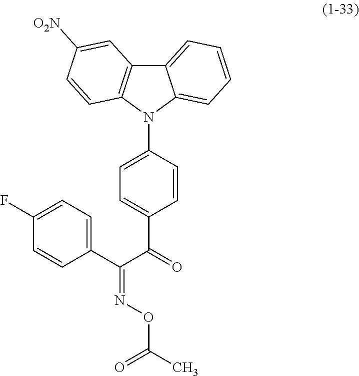 [SMILES: CC(=O)O/N=C(\C(=O)c1ccc(-n2c3ccccc3c3cc([N+](=O)[O-])ccc32)cc1)c1ccc(F)cc1]

Synthesis of the Oxime Ester Phenylcarbazole Compound of Formula 1

There is no restriction on the method for preparing the compound of Formula 1 according to the present invention. For example, the compound of Formula 1 may be synthesized by Reaction Scheme 1:

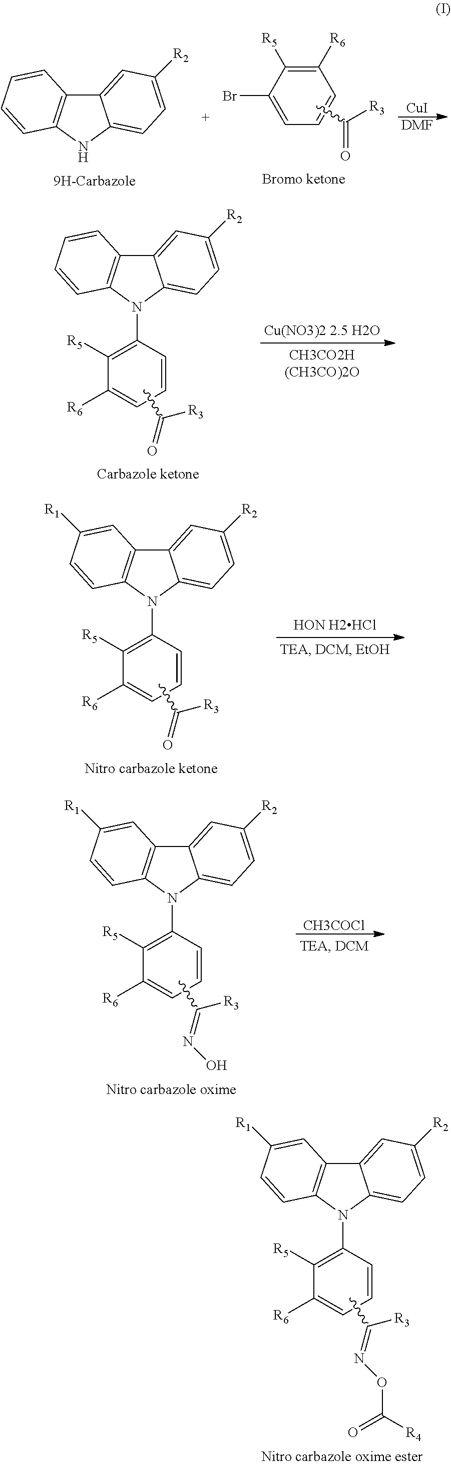

A carbazole ketone compound is prepared by the Ullmann reaction from a 9H-carbazole and a halogen ketone in the presence of copper iodide (CuI). The carbazole ketone compound is nitrated with copper nitrate hemipentahydrate (Cu(NO₃)₂.2.5H₂O) to obtain a nitro carbazole ketone compound. The nitro carbazole ketone compound is aminated with hydroxylamine hydrochloride to obtain a corresponding oxime compound. Next, the nitro carbazole oxime compound is acetylated with acetyl chloride in the presence of triethylamine as a catalyst to yield the oxime ester photoinitiator represented by Formula 1.

The photopolymerizable composition of the present invention includes the oxime ester phenylcarbazole compound as a photoinitiator for photocrosslinking or the oxime ester phenylcarbazole compound represented by Formula 1.

The oxime ester phenylcarbazole compound as a photoinitiator for photocrosslinking or the oxime ester phenylcarbazole compound represented by Formula 1 may be used in combination with one or more known photopolymerization initiators.

In the case where the oxime ester phenylcarbazole compound as a photoinitiator for photocrosslinking or the oxime ester phenylcarbazole compound represented by Formula 1 is used in combination with one or more known photopolymerization initiators, the oxime ester phenylcarbazole compound is preferably present in an amount of 50% by weight or more, based on the total weight of all photopolymerization initiators. The use of the oxime ester phenylcarbazole compound as a photoinitiator for photocrosslinking or the oxime ester phenylcarbazole compound represented by Formula 1 in an amount of 50% by weight or more is effective in increasing the solubility of the photoinitiator while maintaining the sensitivity of the photoinitiator.

As the known photoinitiators, there may be used, for example: acetophenones, including acetophenone, 2,2-diethoxyacetophenone, p-dimethylacetophenone, p-dimethylaminopropiophenone, dichloroacetophenone, trichloroacetophenone, and p-tert-butylacetophenone; benzophenones, including benzophenone, 2-chlorobenzophenone, and p,p′-bisdimethylaminobenzophenone; benzoin ethers, including benzil, benzoin, benzoin methyl ether, benzoin isopropyl ether, and benzoin isobutyl ether; sulfur compounds, including benzyl dimethyl ketal, thioxanthene, 2-chlorothioxanthene, 2,4-diethylthioxanthene, 2-methylthioxanthene, and 2-isopropylthioxanthene; anthraquinones, including 2-ethylanthraquinone, octamethylanthraquinone, 1,2-benzanthraquinone, and 2,3-diphenylanthraquinone; organic peroxides, including azobisisobutyronitrile, benzoyl peroxide, and cumene peroxide; thiol compounds, including 2-mercaptobenzimidazole, 2-mercaptobenzoxazole, and 2-mercaptobenzothiazole; imidazolyl compounds, including 2-(o-chlorophenyl)-4,5-di(m-methoxyphenyl)-imidazolyl dimer; and triazine compounds, including p-methoxytriazine; halomethyl triazine compounds, including 2,4,6-tris(trichloromethyl)-s-triazine, 2-methyl-4,6-bis(trichloromethyl)-s-triazine, 2-[2-(5-methylfuran-2-yl)ethenyl]-4,6-bis(trichloromethyl)-s-triazine, 2-[2-(furan-2-yl)ethenyl]-4,6-bis(trichloromethyl)-s-triazine, 2-[2-4(-diethylamino-2-methylphenyl)ethenyl]-4,6-bis(trichloromethyl)-s-triazine, 2-[2-(3,4-dimethoxyphenol) ethenyl]-4,6-bis(trichloromethyl)-s-triazine, 2-(4-methoxyphenyl)-4,6-bis(trichloromethyl)-s-triazine, 2-(4-ethoxystyryl)-4,6-bis(trichloromethyl)-s-triazine, 2-(4-methoxyphenyl)-4,6-bis(trichloromethyl)-s-triazine, 2-(4-ethoxystyryl)-4,6-bis(trichloromethyl)-s-triazine, and 2-(4-n-butoxyphenyl)-4,6-bis(trichloromethyl)-s-triazine; and aminoketone compounds, including 2-benzyl-2-dimethylamino-1-(4-morpholinophenyl)butan-1-one.

The photopolymerizable composition of the present invention may further include a sensitizer. Examples of sensitizers suitable for use in the photopolymerizable composition of the present invention include: cationic dyes, such as cyanine, xanthene, oxazine, thiazine, diarylmethane, and triarylmethane dyes; neutral dyes, such as merocyanine, coumarin, indigo, aromatic amine, phthalocyanine, azo, quinone, and thioxanthene photosensitive dyes; and other compounds, such as benzophenones, acetophenones, benzoins, thioxantones, anthraquinones, imidazoles, biimidazoles, coumarins, ketocoumarins, triazines, and benzoic acids.

The photopolymerizable composition of the present invention may include a polymeric compound soluble in a solvent or aqueous alkaline solution or a mixture thereof with a photopolymerizable compound having an ethylenically unsaturated bond.

The polymeric compound soluble in a solvent or aqueous alkaline solution can be used as a binder resin. The binder resin may be an acrylic (co)polymer that optionally has an acrylic unsaturated bond in the side chain. The binder resin may be used in an amount ranging from 3 to 50% by weight, based on the total weight of the photopolymerizable composition. This range is preferred because pattern characteristics can be controlled and thin film physical properties such as heat resistance and chemical resistance can be imparted.

For example, the acrylic (co)polymer may have an average molecular weight of 2,000 to 300,000 and a dispersity of 1.0 to 10.0. More preferably, the average molecular weight of the acrylic (co)polymer is from 4,000 to 100,000.

For example, the acrylic (co)polymer may be a (co)polymer of the following monomers: methyl (meth)acrylate, ethyl (meth)acrylate, propyl (meth)acrylate, butyl (meth)acrylate, pentyl (meth)acrylate, hexyl (meth)acrylate, cyclohexyl (meth)acrylate, heptyl (meth)acrylate, octyl (meth)acrylate, nonyl (meth)acrylate, decyl (meth)acrylate, lauryl (meth)acrylate, dodecyl (meth)acrylate, tetradecyl (meth)acrylate, hexadecyl (meth)acrylate, isobornyl (meth)acrylate, adamantyl (meth)acrylate, dicyclopentanyl (meth)acrylate, dicyclopentenyl (meth)acrylate, benzyl (meth)acrylate, 2-methoxyethyl (meth)acrylate, 2-ethoxyethyl (meth)acrylate, acrylic acid, methacrylic acid, itaconic acid, maleic acid, maleic anhydride, monoalkyl maleates, monoalkyl itaconates, monoalkyl fumarates, glycidyl acrylate, glycidyl methacrylate, 3,4-epoxybutyl (meth)acrylate, 2,3-epoxycyclohexyl (meth)acrylate, 3,4-epoxycyclohexyl methyl (meth)acrylate, 3-methyloxetane-3-methyl (meth)acrylate, 3-ethyloxetane-3-methyl (meth)acrylate, styrene, a-methylstyrene, acetoxystyrene, N-methylmaleimide, N-ethylmaleimide, N-propylmaleimide, N-butylmaleimide, N-cyclohexylmaleimide, (meth)acrylamide, and N-methyl (meth)acrylamide. These monomers may be used alone or in combination of two or more thereof.

The acrylic (co)polymer having an acrylic unsaturated bond in the side chain is a (co)polymer prepared by an addition reaction of an acrylic (co)polymer containing a carboxyl group with an epoxy resin at a temperature of 40 to 180° C. Specifically, the acrylic (co)polymer containing a carboxyl group is obtained by copolymerization of an acrylic monomer containing a carboxyl group, such as acrylic acid, methacrylic acid, itaconic acid, maleic acid or a monoalkyl maleate, with two or more monomers selected from alkyl (meth)acrylates, such as methyl (meth)acrylate and hexyl (meth)acrylate, cyclohexyl (meth)acrylate, isobornyl (meth)acrylate, adamantyl (meth)acrylate, dicyclopentanyl (meth)acrylate, dicyclopentenyl (meth)acrylate, benzyl (meth)acrylate, 2-methoxyethyl (meth)acrylate, 2-ethoxyethyl (meth)acrylate, styrene, a-methylstyrene, acetoxystyrene, N-methylmaleimide, N-ethylmaleimide, N-propylmaleimide, N-butylmaleimide, N-cyclohexylmaleimide, (meth)acrylamide, and N-methyl (meth)acrylamide. The epoxy resin is selected from the group consisting of glycidyl acrylate, glycidyl methacrylate, 3,4-epoxybutyl (meth)acrylate, 2,3-epoxycyclohexyl (meth)acrylate, and 3,4-epoxycyclohexylmethyl (meth)acrylate.

Alternatively, the acrylic (co)polymer having an acrylic unsaturated bond in the side chain may be a (co)polymer prepared by an addition reaction of an acrylic (co)polymer containing an epoxy group with an acrylic monomer containing a carboxyl group at a temperature of 40 to 180° C. Specifically, the acrylic (co)polymer containing an epoxy group is obtained by copolymerization of an acrylic monomer containing an epoxy group, such as glycidyl acrylate, glycidyl methacrylate, 3,4-epoxybutyl (meth)acrylate, 2,3-epoxycyclohexyl (meth)acrylate or 3,4-epoxycyclohexylmethyl (meth)acrylate, with two or more monomers selected from alkyl (meth)acrylates, such as methyl (meth)acrylate and hexyl (meth)acrylate, cyclohexyl (meth)acrylate, isobornyl (meth)acrylate, adamantyl (meth)acrylate, dicyclopentanyl (meth)acrylate, dicyclopentenyl (meth)acrylate, benzyl (meth)acrylate, 2-methoxyethyl (meth)acrylate, 2-ethoxyethyl (meth)acrylate, styrene, a-methylstyrene, acetoxystyrene, N-methylmaleimide, N-ethylmaleimide, N-propylmaleimide, N-butylmaleimide, N-cyclohexylmaleimide, (meth)acrylamide, and N-methyl (meth)acrylamide. The acrylic monomer containing a carboxyl group may be selected from the group consisting of acrylic acid, methacrylic acid, itaconic acid, maleic acid, and monoalkyl maleates.

The present invention also provides a photopolymerizable coloring composition including the oxime ester phenylcarbazole compound represented by Formula 1 and a dye or pigment.

The dye or pigment is necessary to apply the composition to the production of a resist for the formation of a color filter or black matrix. Examples of suitable dyes or pigments include cyan, magenta, yellow, and black pigments in the Red, Green, Blue, and subtractive color-mixing systems, such as C.I. Pigment Yellow 12, 13, 14, 17, 20, 24, 55, 83, 86, 93, 109, 110, 117, 125, 137, 139, 147, 148, 153, 154, 166, and 168, C.I. Pigment Orange 36, 43, 51, 55, 59, and 61, C.I. Pigment Red 9, 97, 122, 123, 149, 168, 177, 180, 192, 215, 216, 217, 220, 223, 224, 226, 227, 228, and 240, C.I. Pigment Violet 19, 23, 29, 30, 37, 40, and 50, C.I. Pigment Blue 15, 15:1, 15:4, 15:6, 22, 60, and 64, C.I. Pigment Green 7 and 36, C.I. Pigment Brown 23, 25, and 26, C.I. Pigment Black 7, and titan black.

The present invention also provides a color space, a black matrix, a color filter or a substrate having an organic insulating film formed from the photopolymerizable composition, or a substrate having a film formed by coating with the photopolymerizable composition. For example, the film may be used as a display panel of a TFT-LCD, OLED or TSP.

The photopolymerizable composition may be used to form a pattern. Specifically, a pattern may be formed by applying the photopolymerizable composition onto a substrate to form a layer, removing volatiles, such as solvents, from the layer, exposing the volatile-free layer to light through a photomask, followed by development. The present invention also provides a cured film of the photopolymerizable composition.

The substrate may be, for example, a glass, silicon, polycarbonate, polyester, aromatic polyamide, polyamide-imide, polyimide, aluminum or GaAs substrate whose surface is flat. There is no restriction on the method for applying the photopolymerizable composition onto the substrate. For example, the photopolymerizable composition may be applied onto the substrate by spin coating, casting, roll coating, slit or spin coating. A suitable coater, for example, a spinless coater, may be used to apply the photopolymerizable composition onto the substrate.

Subsequently, volatiles such as solvents are removed by heating. The resulting layer is composed of the solid components of the photopolymerizable composition. The layer is then exposed to light. For example, the layer may be irradiated with active energy rays through a photomask. A low-pressure mercury lamp, medium-pressure mercury lamp, high-pressure mercury lamp, ultrahigh-pressure mercury lamp, xenon lamp or metal halogen lamp is proper as an exposure light source. Laser beams may also be used as active energy rays for exposure.

Electron rays, α rays, β rays, γ rays, X rays, and neutron rays are also possible as the active energy rays. For example, the photomask consists of a glass film and an active energy ray-blocking layer formed on the surface of the glass film.

The active energy rays transmit through a portion of the glass plate in which the light-blocking layer is not formed. The photopolymerizable composition is exposed corresponding to the pattern of the light-transmitting portion. As a result, a non-irradiated region and an irradiated region are created in the exposed layer.

The exposed substrate is developed with an aqueous alkaline solution. For example, the development may be performed by bringing the exposed layer into contact with an aqueous alkaline solution. Specifically, the substrate, on which the photopolymerizable composition layer is formed, may be dipped in an aqueous alkaline solution or may be sprayed with a dilute aqueous alkaline solution. The aqueous alkaline solution may be, for example, an aqueous solution of sodium carbonate, potassium carbonate, sodium hydroxide, potassium hydroxide, tetramethylammonium hydroxide or a basic amine. The non-irradiated region is removed from the photopolymerizable composition layer by the development but the region irradiated with the active energy rays remains unremoved.

The developed substrate is washed and dried by conventional methods. As a result, a desired pattern can be obtained.

For a better understanding of the invention, representative compounds of the present invention will be explained in detail with reference to the following examples, including comparative examples. However, the present invention is not limited to these examples and may be embodied in various different forms.

### Examples 1-9

### Example 1

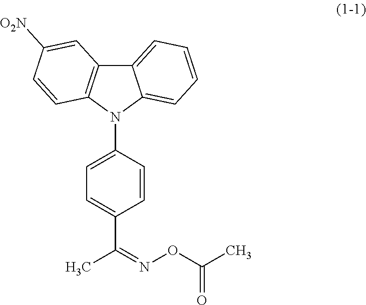 [SMILES: CC(=O)O/N=C(/C)c1ccc(-n2c3ccccc3c3cc([N+](=O)[O-])ccc32)cc1]

### Step 1: Synthesis of 1-(4-(9H-carbazol-9-yl)phenyl)ethanone

A mixture of 9H-carbazole (16.7 g, 100 mmol), 4-bromoacetophenone (25 g, 125 mmol), CuI (2.0 g, 10 mmol), and 18-crown-6 (1.3 g, 5 mmol) was dissolved in dimethylformamide (DMF, 100 mL). The resulting solution was refluxed under a nitrogen atmosphere for 24 h. After completion of the reaction, the reaction mixture was allowed to cool to room temperature. The reaction mixture was poured into 300 mL of water and 200 mL of dimethylene chloride (DMC) was then added thereto. The mixture was stirred vigorously and filtered. The organic layer was separated, dried over Na₂SO₄, evaporated to obtain a brown solid, added with a small amount (50 mL) of acetone, stirred, and filtered to obtain a light brown solid. The solid was dissolved in ethyl acetate (EA) and purified by recrystallization to yield the title compound as a light brown microcrystal. The filtrate was concentrated and left standing at room temperature to obtain a larger amount of the product (overall yield: 26.5 g, 78%).

¹H-NMR (δ, ppm), CDCl₃: 8.20 (d, 2H), 8.15 (d, 2H), 7.70 (d, 2H), 7.49-7.41 (m, 4H), 7.31 (t, 2H), 2.70 (s, 3H)

### Step 2: Synthesis of 1-(4-(3-nitro-9H-carbazol-9-yl)phenyl)ethanone

A solution of the compound prepared in Step 1 (5.7 g, 20 mmol) in methylene chloride (30 mL) was stirred at 0° C., and then a solution of Cu(NO₃)₂.2.5H₂O (5.12 g, 22 mmol) in a mixture of acetic acid (15 mL) and acetic anhydride (30 mL) was added dropwise thereto. The resulting mixture was stirred at room temperature for 1 h. The reaction mixture was poured into distilled water (200 mL) to give a precipitate. The precipitate was collected by filtration, sufficiently washed with water, and dried in air. The crude product was purified by recrystallization from ethyl acetate. For a higher purity of ≥98%, the recrystallized product was purified by silica gel column chromatography using hexane/ethyl acetate (4:1) as the eluent, affording the title compound as a yellow solid (5.8 g, 75.8%).

¹H-NMR (δ, ppm), CDCl₃: 9.06 (d, 1H), 8.35-8.21 (m, 3H), 7.70 (d, 2H), 7.56-7.43 (m, 5H), 2.73 (s, 3H)

### Step 3: Synthesis of (1-(4-(3-nitro-9H-carbazol-9-yl)phenyl)ethanone oxime)

A solution of the compound obtained in Step 2 (2.85 g, 10 mmol) in methylene chloride (15 mL) was added to stirred ethanol (30 mL), and triethylamine (1.01 g, 10 mmol) was then added thereto. To the solution was added hydroxylamine hydrochloride (2.8 g, 30 mmol). The resulting mixture was heated to reflux for 3 h. The reaction solution was cooled to room temperature, poured into 200 mL of cold water, and added with 50 mL of methylene chloride. The organic layer was separated using a separatory funnel and washed three times with distilled water (100 mL) to remove impurities. The organic layer was dried over Na₂SO₄ and the solvent was removed using a rotary evaporator to give an ivory solid. The solid was washed with distilled water and dried under vacuum at 60° C. overnight, affording the title compound in a yield of 90%.

¹H-NMR (δ, ppm), DMSO-d₆: 11.44 (s, 1H), 9.30 (d, 1H), 8.34 (dd, 1H), 8.00 (d, 2H), 7.70 (d, 2H), 7.60-7.50 (m, 3H), 7.47-7.42 (m, 2H), 2.27 (s, 3H)

### Step 4: Synthesis of (1-(4-(3-nitro-9H-carbazol-9-yl)phenyl)ethan-1-one O-acetyl oxime)

The compound obtained in Step 3 (3.0 g, 10 mmol) was dissolved in methylene chloride (20 mL) in a reaction flask protected from light with aluminum foil, and triethylamine (1 mL) was then added thereto. To the mixture was added dropwise acetyl chloride (1.55 g, 20 mmol) with continuous stirring at 0-5° C. After completion of the addition, the resulting mixture was stirred at room temperature overnight. The reaction mixture was poured into distilled water. After stirring for about 10 min, the resulting yellow solid was filtered, dried in air, and recrystallized from methylene chloride/methanol (1/4), affording the final oxime ester of Formula 1-1 as a yellow crystalline solid (yield: 81%). The separation and purification procedures were performed in a yellow room protected from light at ≤420 nm because the oxime ester tends to decompose upon exposure to light.

¹H-NMR (δ, ppm), CDCl₃: 9.06 (s, 1H), 8.32 (d, 1H), 8.20 (d, 1H), 8.05 (d, 2H), 7.62 (d, 2H), 7.53 (t, 1H), 7.43-7.38 (m, 3H), 2.51 (s, 3H), 2.32 (s, 3H)

### Examples 2-9

The compounds shown in Table 1 were synthesized in the same manner as in Example 1, except that the corresponding reactants were used.

TABLE 1

| Example | Formula | | | | | | | |
|---|---|---|---|---|---|---|---|---|
| No. | No. | R₁ | R₂ | R₃ | R₄ | R₅ | R₆ | ¹H-NMR (δ, ppm) |
| 2 | 1-2 | —NO₂ | —H | —C₃H₇ | —CH₃ | —H | —H | 9.05 (d, 1H), 8.32 (dd, 1H), 8.23 |
| | | | | | | | | (d, 1H), 8.03 (d, 2H), 7.62 (d, 2H), |
| | | | | | | | | 7.53 (t, 1H), 7.44-7.40 (m, 3H), |
| | | | | | | | | 2.94 (t, 2H), 2.31 (s, 3H), 1.70-1.47 |
| | | | | | | | | (m, 2H), 0.94 (t, 3H) |
| 3 | 1-3 | —NO₂ | —H | —C₅H₁₁ | —CH₃ | —H | —H | 9.06 (d, 1H), 8.34 (dd, 1H), 8.21 |
| | | | | | | | | (d, 1H), 8.01 (d, 2H), 7.62 (d, 2H), |
| | | | | | | | | 7.53 (t, 1H), 7.44-7.40 (m, 3H), |
| | | | | | | | | 2.94 (t, 2H), 2.31 (s, 3H), 1.69-1.46 |
| | | | | | | | | (m, 2H), 1.44-1.38 (m, 4H), 0.94 (t, |
| | | | | | | | | 3H) |
| 4 | 1-4 | —NO₂ | —H | —C₇H₁₅ | —CH₃ | —H | —H | 9.06 (d, 1H), 8.34 (dd, 1H), 8.21 |
| | | | | | | | | (d, 1H), 8.01 (d, 2H), 7.62 (d, 2H), |
| | | | | | | | | 7.53 (t, 1H), 7.44-7.40 (m, 3H), |
| | | | | | | | | 2.94 (t, 2H), 2.31 (s, 3H), 1.69-1.46 |
| | | | | | | | | (m, 2H), 1.46-1.36 (m, 8H), 0.94 (t, |
| | | | | | | | | 3H) |
| 5 | 1-5 | —NO₂ | —H | 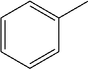 | —CH₃ | —H | —H | 9.08 (d, 1H), 8.32 (dd, 1H), 8.22 (d, 1H), 8.00 (d, 2H), 7.61 (d, 2H), 7.53 (t, 1H), 7.48-7.46 (m, 5H), 7.44-7.40 (m, 3H), 2.31 (s, 3H) |
| 6 | 1-6 | —NO₂ | —H | 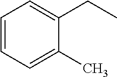 | —CH₃ | —H | —H | 9.08 (d, 1H), 8.34 (dd, 1H), 8.21 (d, 1H), 8.01 (d, 2H), 7.62 (d, 2H), 7.53 (t, 1H), 7.47-7.45 (m, 4H), 7.44-7.40 (m, 3H), 3.70 (s, 2H), 2.31 (s, 3H), 2.28 (s, 3H) |
| 7 | 1-7 | —NO₂ | —H | 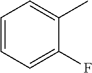 | —CH₃ | —H | —H | 9.05 (d, 1H), 8.31 dd, 1H), 8.21 (d, 1H), 8.01 (d, 2H), 7.62 (d, 2H), 7.53 (t, 1H), 7.52-7.50 (m, 1H), 7.48-7.46 (m, 3H), 7.44-7.40 (m, 3H), 2.31 (s, 3H) |
| 8 | 1-8 | —NO₂ | —H | 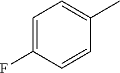 | —CH₃ | —H | —H | 9.04 (d, 1H), 8.32 (dd, 1H), 8.21 (d, 1H), 8.01 (d, 2H), 7.62 (d, 2H), 7.53 (t, 1H), 7.52-7.49 (m, 4H), 7.44-7.40 (m, 3H), 2.31 (s, 3H) |
| 9 | 1-9 | —NO₂ | —H | 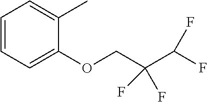 | —CH₃ | —H | —H | 9.04 (d, 1H), 8.31 (dd, 1H), 8.21 (d, 1H), 8.01 (d, 2H), 7.62 (d, 2H), 7.53 (t, 1H), 7.48-7.46 (m, 4H), 7.44-7.40 (m, 3H), 5.80-5.53 (m, 1H), 4.39-4.35 (t, 2H), 2.31 (s, 3H) |

### Comparative Examples 1-2

The compound of Comparative Example 1 shown in Table 2 was synthesized in the same manner as in Example 3, except that Step 2 was omitted and Step 3 proceeded directly.

The compound of Comparative Example 2 shown in Table 2 was synthesized in the same manner as in Example 3, except that Cu(NO₃)₂.2.5H₂O was used in an amount twice that used in Step 2.

TABLE 2

| Comparative | Formula | | | | | | | |
|---|---|---|---|---|---|---|---|---|
| Example No. | No. | R₁ | R₂ | R₃ | R₄ | R₅ | R₆ | ¹H—NMR (δ, ppm) |
| 1 | 1-34 | —H | —H | —C₅H₁₁ | —CH₃ | —H | —H | 9.06 (s, 1H), 8.32 (d, 1H), |
| | | | | | | | | 8.20 (d, 1H), 8.05 (d, 2H), |
| | | | | | | | | 7.62 (d, 2H), 7.53 (t, 1H), |
| | | | | | | | | 7.43-7.38 (m, 3H), 2.89 (t, |
| | | | | | | | | 2H), 2.30 (s, 3H), 1.67-1.45 |
| | | | | | | | | (m, 2H), 1.43-1.36 (m, 4H), |
| | | | | | | | | 0.94 (t, 3H) |
| 2 | 1-35 | —NO₂ | —NO₂ | —C₅H₁₁ | —CH₃ | —H | —H | 9.57 (s, 1H), 9.29 (s, 1H), |
| | | | | | | | | 8.64 (d, 1H), 8.31 (d, 1H), |
| | | | | | | | | 8.12 (d, 1H), 7.85 (d, 1H), |
| | | | | | | | | 7.83 (d, 1h), 7.70-7.47 (m, |
| | | | | | | | | 3H), 3.01 (t, 2H), 2.34 (s, |
| | | | | | | | | 3H), 1.72-1.49 (m, 2H), 1.46- |
| | | | | | | | | 1.39 (m, 4H), 0.96 (t, 3H) |

For reference, the structures of Comparative Examples 1 and 2 are represented by Formulae 1-34 and 1-35, respectively.

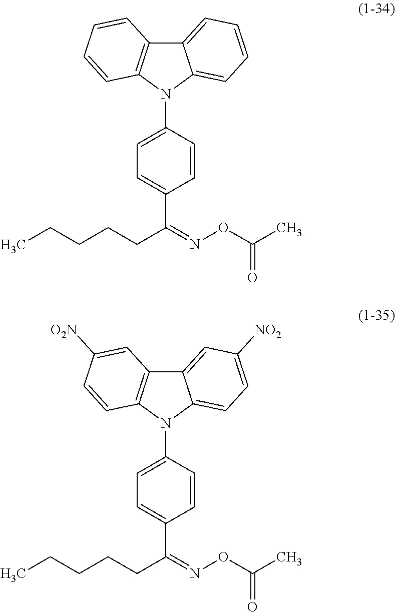

### Examples 10-18

### Example 10

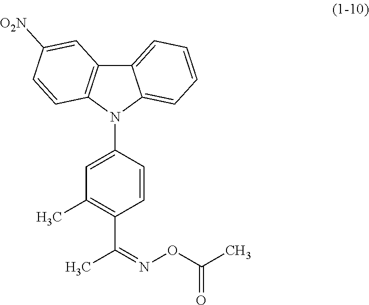 [SMILES: CC(=O)O/N=C(/C)c1ccc(-n2c3ccccc3c3cc([N+](=O)[O-])ccc32)cc1C]

### Step 1: Synthesis of 1-(4-(9H-carbazol-9-yl)-2-methylphenyl)ethanone

A mixture of 9H-carbazole (16.7 g, 100 mmol), 1-(4-bromo-2-methylphenyl)ethanone (26.6 g, 125 mmol), CuI (2.0 g, 10 mmol), and 18-crown-6 (1.3 g, 5 mmol) was dissolved in dimethylformamide (DMF, 100 mL). The resulting solution was refluxed under a nitrogen atmosphere for 24 h. After completion of the reaction, the reaction mixture was allowed to cool to room temperature. The reaction mixture was poured into 300 mL of water and 200 mL of methylene chloride (MC) was then added thereto. The mixture was stirred vigorously and filtered. The organic layer was separated, dried over Na₂SO₄, evaporated to obtain a brown solid, added with a small amount (50 mL) of acetone, stirred, and filtered to obtain a light brown solid. The solid was purified by silica gel column chromatography to isolate the title compound from its other isomers (overall yield: 15.7 g, 52%).

¹H-NMR (δ, ppm), CDCl₃: 8.20 (d, 2H), 8.14 (d, 2H), 7.68 (d, 2H), 7.48-7.40 (m, 3H), 7.32 (t, 2H), 2.70 (s, 3H), 2.68 (s, 3H)

### Step 2: Synthesis of 1-(4-(3-nitro-9H-carbazol-9-yl)phenyl)ethanone

A solution of the compound prepared in Step 1 (12.0 g, 40 mmol) in methylene chloride (60 mL) was stirred at 0° C., and then a solution of Cu(NO₃)₂.2.5H₂O (10.2 g, 44 mmol) in a mixture of acetic acid (30 mL) and acetic anhydride (60 mL) was added dropwise thereto. The resulting mixture was stirred at room temperature for 1 h. The reaction mixture was poured into distilled water (400 mL) to give a precipitate. The precipitate was collected by filtration, sufficiently washed with water, and dried in air. The crude product was purified by recrystallization from ethyl acetate. For a higher purity of ≥98%, the recrystallized product was purified by silica gel column chromatography using hexane/ethyl acetate (4:1) as the eluent, affording the title compound as a yellow solid (10.2 g, 74.1%).

¹H-NMR (δ, ppm), CDCl₃: 9.06 (d, 1H), 8.34-8.20 (m, 3H), 7.70 (d, 2H), 7.50-7.32 (m, 5H), 2.80 (s, 3H), 2.73 (s, 3H)

### Step 3: Synthesis of 1-(4-(3-nitro-9H-carbazol-9-yl)phenyl)ethanone oxime

A solution of the compound obtained in Step 2 (8.0 g, 23.2 mmol) in methylene chloride (40 mL) was added to stirred ethanol (80 mL), and triethylamine (2.36 g, 23.2 mmol) was then added thereto. To the solution was added hydroxylamine hydrochloride (4.8 g, 69.6 mmol). The resulting mixture was heated to reflux for 3 h. The reaction solution was cooled to room temperature, poured into 600 mL of cold water, and added with 150 mL of methylene chloride. The organic layer was separated using a separatory funnel and washed three times with distilled water (350 mL) to remove impurities. The organic layer was dried over Na₂SO₄ and the solvent was removed using a rotary evaporator to give an ivory solid. The solid was washed with distilled water and dried under vacuum at 60° C. overnight, affording the title compound in a yield of 89%.

¹H-NMR (δ, ppm), DMSO-d₆: 11.44 (s, 1H), 9.30 (d, 1H), 8.32 (dd, 1H), 8.00 (d, 2H), 7.68 (d, 2H), 7.61-7.53 (m, 2H), 7.46-7.42 (m, 2H), 2.43 (s, 2H), 2.27 (s, 3H)

### Step 4: Synthesis of 1-(4-(3-nitro-9H-carbazol-9-yl)phenyl)ethanone O-acetyl oxime

The compound obtained in Step 3 (6.0 g, 20 mmol) was dissolved in methylene chloride (40 mL) in a reaction flask protected from light with aluminum foil, and triethylamine (1 mL) was then added thereto. To the mixture was added dropwise acetyl chloride (3.1 g, 40 mmol) with continuous stirring at 0-5° C. After completion of the addition, the resulting mixture was stirred at room temperature overnight. The reaction mixture was poured into distilled water. After stirring for about 10 min, the resulting yellow solid was filtered, dried in air, and recrystallized from methylene chloride/methanol (1/4), affording the final oxime ester of Formula 1-10 as a yellow crystalline solid (yield: 82%). The separation and purification procedures were performed in a yellow room protected from light at ≤420 nm because the oxime ester tends to decompose upon exposure to light.

¹H-NMR (δ, ppm), CDCl₃: 9.06 (s, 1H), 8.32 (d, 1H), 8.21 (d, 1H), 8.04 (d, 2H), 7.62 (d, 2H), 7.53 (t, 1H), 7.40-7.36 (m, 3H), 2.51 (s, 3H), 2.30 (s, 3H), 2.32 (s, 3H)

### Examples 11-18

The compounds shown in Table 3 were synthesized in the same manner as in Example 10, except that the corresponding reactants were used.

TABLE 3

| Example | Formula | | | | | | | |
|---|---|---|---|---|---|---|---|---|
| No. | No. | R₁ | R₂ | R₃ | R₄ | R₅ | R₆ | ¹H-NMR (δ, ppm) |
| 11 | 1-11 | —NO₂ | —H | —C₃H₇ | —CH₃ | —H | —CH₃ | 9.05 (d, 1H), 8.32 (dd, 1H), 8.23 |
| | | | | | | | | (d, 1H), 8.03 (d, 2H), 7.62 (d, 2H), |
| | | | | | | | | 7.51 (d, 1H), 7.42-7.40 (m, 2H), |
| | | | | | | | | 2.94 (t, 2H), 2.52 (s, 3H), 2.31 (s, |
| | | | | | | | | 3H), 1.70-1.47 (m, 2H), 0.94 (t, |
| | | | | | | | | 3H) |
| 12 | 1-12 | —NO₂ | —H | —C₅H₁₁ | —CH₃ | —H | —CH₃ | 9.06 (d, 1H), 8.34 (dd, 1H), 8.21 |
| | | | | | | | | (d, 1H), 8.01 (d, 2H), 7.62 (d, 2H), |
| | | | | | | | | 7.53 (d, 1H), 7.43-7.40 (m, 2H), |
| | | | | | | | | 2.94 (t, 2H), 2.51 (s, 3H), 2.31 (s, |
| | | | | | | | | 3H), 1.69-1.46 (m, 2H), 1.44-1.38 |
| | | | | | | | | (m, 4H), 0.94 (t, 3H) |
| 13 | 1-13 | —NO₂ | —H | —C₇H₁₅ | —CH₃ | —H | —CH₃ | 9.06 (d, 1H), 8.34 (dd, 1H), 8.21 |
| | | | | | | | | (d, 1H), 8.01 (d, 2H), 7.62 (d, 2H), |
| | | | | | | | | 7.53 (d, 1H), 7.44-7.40 (m, 2H), |
| | | | | | | | | 2.94 (t, 2H), 2.54 (s, 3H), 2.31 (s, |
| | | | | | | | | 3H), 1.69-1.46 (m, 2H), 1.46-1.36 |
| | | | | | | | | (m, 8H), 0.94 (t, 3H) |
| 14 | 1-14 | —NO₂ | —H | 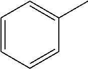 | —CH₃ | —H | —CH₃ | 9.08 (d, 1H), 8.32 (dd, 1H), 8.22 (d, 1H), 8.00 (d, 2H), 7.61 (d, 2H), 7.53 (t, 1H), 7.48-7.46 (m, 4H), 7.44-7.40 (m, 3H), 2.53 (s, 3H), 2.31 (s, 3H) |
| 15 | 1-15 | —NO₂ | —H | 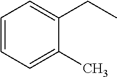 | —CH₃ | —H | —CH₃ | 9.07 (d, 1H), 8.34 (dd, 1H), 8.21 (d, 1H), 8.01 (d, 2H), 7.62 (d, 2H), 7.53 (d, 1H), 7.49-7.43 (m, 4H), 7.42-7.39 (m, 2H), 3.70 (s, 2H), 2.43 (s, 3H), 2.31 (s, 3H), 2.28 (s, |
| | | | | | | | | 3H) |
| 16 | 1-16 | —NO₂ | —H | 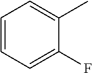 | —CH₃ | —H | —CH₃ | 9.04 (d, 1H), 8.31 (dd, 1H), 8.21 (d, 1H), 8.01 (d, 2H), 7.63 (d, 2H), 7.60-7.56 (m, 2H), 7.52-7.50 (m, 1H), 7.48-7.46 (m, 2H), 7.44-7.40 (m, 2H), 2.48 (s, 3H), 2.31 (s, 3H) |
| 17 | 1-17 | —NO₂ | —H | 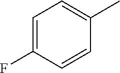 | —CH₃ | —H | —CH₃ | 9.04 (d, 1H), 8.32 (dd, 1H), 8.24 (d, 1H), 8.01 (d, 2H), 7.62 (d, 2H), 7.53 (t, 1H), 7.52-7.49 (m, 2H), 7.44-7.40 (m, 4H), 2.47 (s, 3H), 2.31 (s, 3H) |
| 18 | 1-18 | —NO₂ | —H | 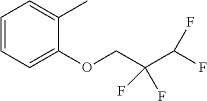 | —CH₃ | —H | —CH₃ | 9.04 (d, 1H), 8.31 (dd, 1H), 8.21 (d, 1H), 8.01 (d, 2H), 7.62 (d, 2H), 7.53-7.48 (m, 4H), 7.43-7.39 (m, 3H), 5.80-5.53 (m, 1H), 4.39-4.35 (t, 2H), 2.48 (s, 3H), 2.31 (s, 3H) |

### Examples 19-27

### Example 19

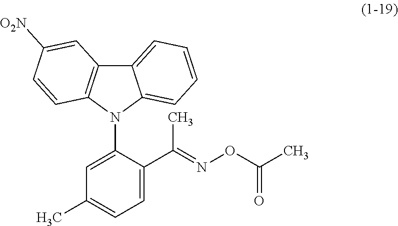 [SMILES: CC(=O)O/N=C(\C)c1ccc(C)cc1-n1c2ccccc2c2cc([N+](=O)[O-])ccc21]

### Step 1: Synthesis of 1-(2-(9H-carbazol-9-yl)-4-methylphenyl)ethanone

A mixture of 9H-carbazole (16.7 g, 100 mmol), 3-bromo-5-methylacetophenone 1-2-bromo-4-methylphenyl)ethanone (26.6 g, 125 mmol), CuI (2.0 g, 10 mmol), and 18-crown-6 (1.3 g, 0.50 mmol) was dissolved in dimethylformamide (DMF, 100 mL). The resulting solution was refluxed under a nitrogen atmosphere for 24 h. After completion of the reaction, the reaction mixture was allowed to cool to room temperature. The reaction mixture was poured into 300 mL of water and 200 mL of methylene chloride (MC) was then added thereto. The mixture was stirred vigorously and filtered. The organic layer was separated, dried over Na₂SO₄, evaporated to obtain a brown solid, added with a small amount (50 mL) of acetone, stirred, and filtered to obtain a light brown solid. The solid was purified by silica gel column chromatography to isolate the title compound from its other isomers (overall yield: 7.5 g, 25%).

¹H-NMR (δ, ppm), CDCl₃: 8.22 (d, 2H), 8.16 (d, 2H), 7.70 (d, 2H), 7.50-7.41 (m, 3H), 7.31 (t, 2H), 2.70 (s, 3H), 2.42 (s, 3H)

### Step 2: Synthesis of 1-(4-methyl-2-(3-nitro-9H-carbazol-9-yl)phenyl)ethanone

A solution of the compound prepared in Step 1 (10 g, 33.4 mmol) in methylene chloride (60 mL) was stirred at 0° C., and then a solution of Cu(NO₃)₂.2.5H₂O (8.55, 36.7 mmol) in a mixture of acetic acid (30 mL) and acetic anhydride (60 mL) was added dropwise thereto. The resulting mixture was stirred at room temperature for 1 h. The reaction mixture was poured into distilled water (400 mL) to give a precipitate. The precipitate was collected by filtration, sufficiently washed with water, and dried in air. The crude product was purified by recrystallization from ethyl acetate. For a higher purity of ≥98%, the recrystallized product was purified by silica gel column chromatography using hexane/ethyl acetate (4:1) as the eluent, affording the title compound as a yellow solid (8.7 g, 75.6%).

¹H-NMR (δ, ppm), CDCl₃: 9.05 (d, 1H), 8.34-8.21 (m, 3H), 7.68 (d, 2H), 7.50-7.38 (m, 4H), 2.73 (s, 3H), 2.31 (s, 3H)

### Step 3: Synthesis of 1-(4-methyl-2-(3-nitro-9H-carbazol-9-yl)phenyl)ethanone oxime

A solution of the compound obtained in Step 2 (5 g, 13.9 mmol) in methylene chloride (30 mL) was added to stirred ethanol (60 mL), and triethylamine (1.41 g, 13.9 mmol) was then added thereto. To the solution was added hydroxylamine hydrochloride (2.89 g, 41.7 mmol). The resulting mixture was heated to reflux for 3 h. The reaction solution was cooled to room temperature, poured into 400 mL of cold water, and added with 100 mL of methylene chloride. The organic layer was separated using a separatory funnel and washed three times with distilled water (200 mL) to remove impurities. The organic layer was dried over Na₂SO₄ and the solvent was removed using a rotary evaporator to give an ivory solid. The solid was washed with distilled water and dried under vacuum at 60° C. overnight, affording the title compound in a yield of 85%.

¹H-NMR (δ, ppm), DMSO-d₆: 11.40 (s, 1H), 9.28 (d, 1H), 8.30 (dd, 1H), 8.00 (d, 2H), 7.68 (d, 2H), 7.58-7.50 (m, 2H), 7.47-7.42 (m, 2H), 2.70 (s, 3H), 2.27 (s, 3H)

### Step 4: Synthesis of 1-(4-methyl-2-(3-nitro-9H-carbazol-9-yl)phenyl)ethanone O-acetyl oxime

The compound obtained in Step 3 (4 g, 11.1 mmol) was dissolved in methylene chloride (30 mL) in a reaction flask protected from light with aluminum foil, and triethylamine (1.5 mL) was then added thereto. To the mixture was added dropwise acetyl chloride (1.75 g, 22.3 mmol) with continuous stirring at 0-5° C. After completion of the addition, the resulting mixture was stirred at room temperature overnight. The reaction mixture was poured into distilled water. After stirring for about 10 min, the resulting yellow solid was filtered, dried in air, and recrystallized from methylene chloride/methanol (1/4), affording the final oxime ester of Formula 1-19 as a yellow crystalline solid (yield: 77%). The separation and purification procedures were performed in a yellow room protected from light at ≤420 nm because the oxime ester tends to decompose upon exposure to light.

¹H-NMR (δ, ppm), CDCl₃: 9.06 (s, 1H), 8.31 (d, 1H), 8.22 (d, 1H), 8.04 (d, 2H), 7.64 (d, 2H), 7.52 (t, 1H), 7.44-7.38 (m, 3H), 2.40 (s, 3H), 2.32 (s, 3H), 2.28 (s, 3H)

### Examples 20-27

The compounds shown in Table 4 were synthesized in the same manner as in Example 19, except that the corresponding reactants were used.

TABLE 4

| Example | Formula | | | | | | | |
|---|---|---|---|---|---|---|---|---|
| No. | No. | R₁ | R₂ | R₃ | R₄ | R₅ | R₆ | ¹H-NMR (δ, ppm) |
| 20 | 1-20 | —NO₂ | —H | —C₃H₇ | —CH₃ | —H | —CH₃ | 9.05 (d, 1H), 8.32 (dd, 1H), 8.23 |
| | | | | | | | | (d, 1H), 8.03 (d, 2H), 7.62 (d, 1H), |
| | | | | | | | | 7.55-7.48 (m, 2H), 7.42-7.40 (m, |
| | | | | | | | | 2H), 2.93 (t, 2H), 2.40 (s, 3H), 2.31 |
| | | | | | | | | (s, 3H), 1.70-1.47 (m, 2H), 0.94 (t, |
| | | | | | | | | 3H) |
| 21 | 1-21 | —NO₂ | —H | —C₅H₁₁ | —CH₃ | —H | —CH₃ | 9.06 (d, 1H), 8.34 (dd, 1H), 8.21 |
| | | | | | | | | (d, 1H), 8.01 (d, 2H), 7.60 (d, 1H), |
| | | | | | | | | 7.55-7.48 (m, 2H), 7.43-7.40 (m, |
| | | | | | | | | 2H), 2.94 (t, 2H), 2.51 (s, 3H), 2.31 |
| | | | | | | | | (s, 3H), 1.69-1.46 (m, 2H), 1.44- |
| | | | | | | | | 1.38 (m, 4H), 0.94 (t, 3H) |
| 22 | 1-22 | —NO₂ | —H | —C₇H₁₅ | —CH₃ | —H | —CH₃ | 9.06 (d, 1H), 8.34 (dd, 1H), 8.21 |
| | | | | | | | | (d, 1H), 8.00 (d, 2H), 7.62 (d, 2H), |
| | | | | | | | | 7.53 (d, 1H), 7.43-7.40 (m, 2H), |
| | | | | | | | | 2.94 (d, 2H), 2.54 (s, 3H), 2.31 (s, |
| | | | | | | | | 3H), 1.70-1.65 (m, 2H), 1.52-1.36 |
| | | | | | | | | (m, 8H), 0.93 (t, 3H) |
| 23 | 1-23 | —NO₂ | —H | 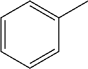 | —CH₃ | —H | —CH₃ | 9.07 (d, 1H), 8.32 (dd, 1H), 8.22 (d, 1H), 8.00 (d, 2H), 7.61d, 2H), 7.53 (t, 1H), 7.50-7.46 (m, 4H), 7.44-7.40 (m, 3H), 2.41 (s, 3H), 2.31 (s, 3H) |
| 24 | 1-24 | —NO₂ | —H | 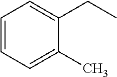 | —CH₃ | —H | —CH₃ | 9.07 (d, 1H), 8.33 (dd, 1H), 8.21 (d, 1H), 8.01 (d, 2H), 7.62 (d, 2H), 7.53 (d, 1H), 7.48-7.43 (m, 4H), 7.41-7.38 (m, 2H), 3.72 (s, 2H), 2.43 (s, 3H), 2.31 (s, 3H), 2.28 (s, |
| | | | | | | | | 3H) |
| 25 | 1-25 | —NO₂ | —H | 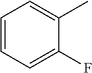 | —CH₃ | —H | —CH₃ | 9.04 (d, 1H), 8.31 (dd, 1H), 8.20 (d, 1H), 7.99 (d, 2H), 7.70 (d, 2H), 7.60-7.55 (m, 2H), 7.52-7.50 (m, 1H), 7.48-7.46 (m, 2H), 7.44-7.40 (m, 2H), 2.47 (s, 3H), 2.31 (s, 3H) |
| 26 | 1-26 | —NO₂ | —H | 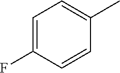 | —CH₃ | —H | —CH₃ | 9.04 (d, 1H), 8.32 (dd, 1H), 8.24 (d, 1H), 8.01 (d, 2H), 7.62 (d, 2H), 7.52-7.49 (m, 3H), 7.44-7.40 (m, 4H), 2.46 (s, 3H), 2.31 (s, 3H) |
| 27 | 1-27 | —NO₂ | —H | 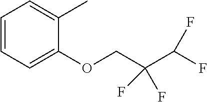 | —CH₃ | —H | —CH₃ | 9.04 (d, 1H), 8.30 (dd, 1H), 8.21 (d, 1H), 8.01 (d, 2H), 7.61 (d, 2H), 7.53-7.48 (m, 4H), 7.43-7.39 (m, 3H), 5.80-5.53 (m, 1H), 4.40-4.35 (t, 2H), 2.48 (s, 3H), 2.31 (s, 3H) |

### Examples 28-33

### Example 28

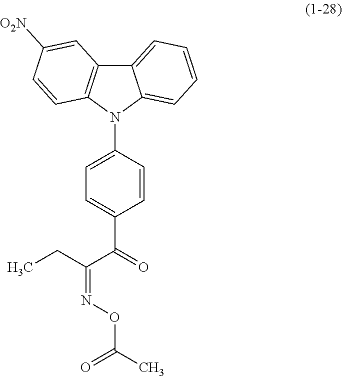 [SMILES: CC/C(=N/OC(C)=O)C(=O)c1ccc(-n2c3ccccc3c3cc([N+](=O)[O-])ccc32)cc1]

### Step 1: Synthesis of 1-(4-(9H-carbazol-9-yl)phenyl)butan-1-one

A mixture of 9H-carbazole (16.7 g, 100 mmol), 1-(4-bromophenyl)butan-1-one (28 g, 125 mmol), CuI (2.0 g, 10 mmol), and 18-crown-6 (1.3 g, 0.50 mmol) was dissolved in dimethylformamide (DMF, 100 mL). The resulting solution was refluxed under a nitrogen atmosphere for 24 h. After completion of the reaction, the reaction mixture was allowed to cool to room temperature. The reaction mixture was poured into 300 mL of water and 200 mL of methylene chloride (DMC) was then added thereto. The mixture was stirred vigorously and filtered. The organic layer was separated, dried over Na₂SO₄, evaporated to obtain a brown solid, added with a small amount (50 mL) of acetone, stirred, and filtered to obtain a light brown solid. The solid was dissolved in ethyl acetate (EA) and purified by recrystallization to yield the title compound as a light brown microcrystal. The filtrate was concentrated and left standing at room temperature to obtain a larger amount of the product (overall yield: 26 g, 76.2%).

¹H-NMR (δ, ppm), CDCl₃: 8.20 (d, 2H), 8.15 (d, 2H), 7.70 (d, 2H), 7.49-7.41 (m, 4H), 7.31 (t, 2H), 2.31 (t, 2H), 1.80 (m, 2H), 1.29 (t, 3H)

### Step 2: Synthesis of 1-(4-(3-nitro-9H-carbazol-9-yl)phenyl)butan-1-one

A solution of the compound prepared in Step 1 (10 g, 29.3 mmol) in methylene chloride (60 mL) was stirred at 0° C., and then a solution of Cu(NO₃)₂.2.5H₂O (7.49 g, 32.2 mmol) in a mixture of acetic acid (30 mL) and acetic anhydride (60 mL) was added dropwise thereto. The resulting mixture was stirred at room temperature for 1 h. The reaction mixture was poured into distilled water (400 mL) to give a precipitate. The precipitate was collected by filtration, sufficiently washed with water, and dried in air. The crude product was purified by recrystallization from ethyl acetate. For a higher purity of ≥98%, the recrystallized product was purified by silica gel column chromatography using hexane/ethyl acetate (4:1) as the eluent, affording the title compound as a yellow solid (8.8 g, 77.8%).

¹H-NMR (δ, ppm), CDCl₃: 9.06 (d, 1H), 8.35-8.21 (m, 3H), 7.70 (d, 2H), 7.56-7.43 (m, 5H), 2.69 (t, 2H), 1.82 (m, 2H), 1.31 (t, 3H)

### Step 3: Synthesis of 2-(hydroxyimino)-1-(4-(3-nitro-9H-carbazol-9-yl)phenyl)butan-1-one

The compound obtained in Step 2 (5 g, 12.9 mmol) was dissolved in dimethylformamide (DMF, 30 mL) and then isopentyl nitrate (IPN) (1.81 g, 15.5 mmol) was added thereto. The mixture was stirred at room temperature for 12 h. The reaction was stopped by the addition of HCl (0.12 g, 3.22 mmol). 100 mL of distilled water was poured into the reaction mixture and 100 mL of ethyl acetate (EA) was added thereto. The organic layer was separated using a separatory funnel and washed three times with distilled water (100 mL) to remove impurities. The organic layer was dried over Na₂SO₄ and the solvent was removed using a rotary evaporator to give an ivory solid. The solid was washed with distilled water and dried under vacuum at 60° C. overnight, affording the title compound in a yield of 76.6%.

¹H-NMR (δ, ppm), DMSO-d₆: 11.20 (s, 1H), 9.30 (d, 1H), 8.32 (dd, 1H), 8.21 (d, 1H) (m, 1H), 8.00 (d, 1H), 7.70 (d, 1H), 7.60-7.50 (m, 2H), 7.47-7.42 (m, 2H), 2.52 (q, 2H), 1.34 (t, 3H)

### Step 4: Synthesis of 2-(acetoxyimino)-1-(4-(3-nitro-9H-carbazol-9-yl)phenyl)butan-1-one

The compound obtained in Step 3 (3.0 g, 7.22 mmol) was dissolved in methylene chloride (20 mL) in a reaction flask protected from light with aluminum foil, and triethylamine (1 mL) was then added thereto. To the mixture was added dropwise acetyl chloride (1.13 g, 14.4 mmol) with continuous stirring at 0-5° C. After completion of the addition, the resulting mixture was stirred at room temperature overnight. The reaction mixture was poured into distilled water. After stirring for about 10 min, the resulting yellow solid was filtered, dried in air, and recrystallized from methylene chloride/methanol (1/4), affording the final oxime ester of Formula 1-28 as a yellow crystalline solid (yield: 75%). The separation and purification procedures were performed in a yellow room protected from light at ≤420 nm because the oxime ester tends to decompose upon exposure to light.

¹H-NMR (δ, ppm), CDCl₃: 9.06 (s, 1H), 8.32 (d, 1H), 8.20 (d, 1H), 8.04 (d, 2H), 7.60 (d, 2H), 7.51 (t, 1H), 7.43-7.38 (m, 3H), 2.35 (s, 3H), 2.32 (q, 2H), 1.32 (t, 3H)

### Examples 29-33

The compounds shown in Table 5 were synthesized in the same manner as in Example 28, except that the corresponding reactants were used.

TABLE 5

| Example | Formula | | | | | | | |
|---|---|---|---|---|---|---|---|---|
| No. | No. | R₁ | R₂ | R₃ | R₄ | R₅ | R₆ | ¹H-NMR (δ, ppm) |
| 29 | 1-29 | —NO₂ | —H | —C₅H₁₁ | —CH₃ | —H | —H | 9.05 (d, 1H), 8.32 (dd, 1H), 8.22 (d, |
| | | | | | | | | 1H), 8.03 (d, 2H), 7.72 (d, 2H), 7.52 (t, |
| | | | | | | | | 1H), 7.43-7.38 (m, 3H), 2.30 (s, 3H), |
| | | | | | | | | 2.28 (t, 3H), 1.60 (m, 4H), 0.93 (t, 3H) |
| 30 | 1-30 | —NO₂ | —H | —C₇H₁₅ | —CH₃ | —H | —H | 9.06 (d, 1H), 8.34 (dd, 1H), 8.21 (d, |
| | | | | | | | | 1H), 8.01 (d, 2H), 7.62 (d, 2H), 7.52 (t, |
| | | | | | | | | 1H), 7.44-7.40 (m, 3H), 2.94 (t, 2H), |
| | | | | | | | | 2.31 (s, 3H), 2.29 (t, 3H), 1.66-1.36 (m, |
| | | | | | | | | 10H), 0.94 (t, 3H) |
| 31 | 1-31 | —NO₂ | —H | 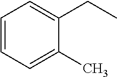 | —CH₃ | —H | —H | 9.07(d, 1H), 8.34 (dd, 1H), 8.30 (d, 1H), 8.12 (d, 2H), 7.80 (d, 2H), 7.62 (t, 1H), 7.47-7.45 (m, 4H), 7.44-7.40 (m, 3H), 3.80 (s, 2H), 2.32 (s, 3H), 2.28 (s, 3H) |
| 32 | 1-32 | —NO₂ | —H | 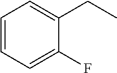 | —CH₃ | —H | —H | 9.04(d, 1H), 8.31 (dd, 1H), 8.21 (d, 1H), 8.01 (d, 2H), 7.80 (d, 2H), 7.68 (t, 1H), 7.54-7.50 (m, 1H), 7.48-7.46 (m, 3H), 7.44-7.40 (m, 3H), 3.79 (s, 2H), 2.30 (s, 3H), 2.27 (s, 3H) |
| 33 | 1-33 | —NO₂ | —H | 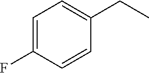 | —CH₃ | —H | —H | 9.04(d, 1H), 8.32 dd, 1H), 8.21 (d, 1H), 8.00 (d, 2H), 7.62 (d, 2H), 7.56 (t, 1H), 7.52-7.48 (m, 4H), 7.44-7.37 (m, 3H), 3.78 (s, 2H) 2.26 (s, 3H) |

OXE-01 and OXE-02 commercially available from BASF were prepared as photoinitiators of Comparative Examples 3 and 4, respectively.

### Additional Examples 1-33 and Additional Comparative Examples 1-4

Transparent resist compositions and black resist compositions were prepared using the compounds of Examples 1-33 and Comparative Examples 1-4 by the following respective procedures.

<Transparent Resist Compositions>

17 g of an acrylic binder resin, 13.6 g of dipentaerythritol hexaacrylate, 1.5 g of each of the compounds of Examples 1-33 and Comparative Examples 1-4, 67 g of propylene glycol monoethyl ether, and 500 ppm of a surfactant (FC-430, 3M) were sufficiently stirred to prepare a transparent photosensitive resist composition.

<Black Resist Compositions>

30 g of carbon black, 20 g of titan black, 13 g of a polyester binder resin, 10 g of dipentaerythritol hexaacrylate, 2.5 g of each of the compounds of Examples 1-33 and Comparative Examples 1-4, 300 g of propylene glycol monoethyl ether, and 500 ppm of a surfactant (FC-430, 3M) were mixed together to prepare a black photosensitive resist composition.

<Evaluation of Physical Properties>

The transparent photosensitive resist compositions were evaluated as follows.

Each of the photosensitive compositions was spin coated on 4-inch circular glass at 800-900 rpm for 15 s and dried on a hot plate at 90° C. for 100 s. The coating was exposed to light from an ultrahigh-pressure mercury lamp as a light source through a predetermined mask, developed by spraying with 0.04% potassium hydroxide solution at 25° C. for 60 s, and washed with water to clean its surface.

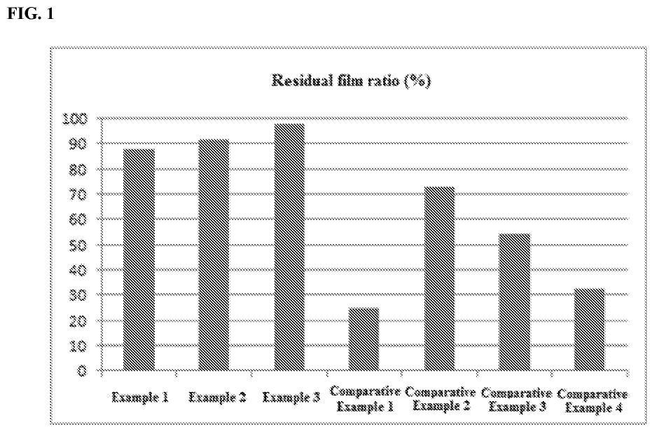

After drying, the coating was baked at 230° C. for 40 min to obtain a pattern. The physical properties of the composition were evaluated by the following respective procedures. The results are shown in Table 6 and FIG. 1.

(1) Adhesiveness

After cross-cuts were scribed in the form of grids, a peeling test was conducted using a cellophane tape. The peeling state of the cross-cuts was observed. Adhesiveness was judged to be “∘” when no cross-cut was peeled and “X” when one or more cross-cuts were peeled.

(2) Sensitivity

Each of the photopolymerizable compositions was spin coated on 4-inch circular glass and dried at 100° C. for 90 s. The coating was exposed to light from an ultrahigh-pressure mercury lamp as a light source through patterned masks with different transmittances, developed by spraying with 0.04% potassium hydroxide solution at 25° C. for 60 s, and washed with water to clean its surface. The thicknesses of each pattern before and after development were measured using a contact-type thickness meter. Sensitivity was defined as the exposure dose at which the thickness of the patterned coating after development reached 80% of the thickness of the coating before development.

(3) Residual Film Ratio

Each of the photopolymerizable compositions was spin coated on 4-inch circular glass and dried at 100° C. for 90 s. The coating was exposed to light through a predetermined mask, developed by spraying with 0.04% potassium hydroxide solution at 25° C. for 60 s, and washed with water to clean its surface. The thicknesses of each coating before and after development were measured using a contact-type thickness meter. Residual film ratio was defined as the ratio (%) of the thickness of the coating after development to the thickness of the coating before development.

(4) Pattern Stability

A hole pattern of each of the photopolymerizable compositions was formed on a silicon wafer. The hole pattern was cut in the vertical direction and the cross-section of the pattern was observed under an electron microscope. The stability of the pattern was judged to “∘” when the side wall of the pattern stood at an angle of 55° with respect to the substrate without film loss and “X” when film loss was observed.

(5) Chemical Resistance

Each of the photopolymerizable compositions was spin coated on a silicon wafer, followed by a series of pre-baking, exposure, development, and post-baking to form a resist film. The resist film was immersed in a stripper solution at 40° C. for 10 min. Variations in the transmittance and thickness of the resist film were investigated. Chemical resistance was judged to be “∘” when the transmittance and thickness variations were <2% and “X” when the transmittance and thickness variations were ≥2%.

(6) Whitening

Each of the photopolymerizable compositions was spin coated on a substrate, followed by pre-baking to form a film. Whitening resistance was judged to be “∘” when the surface of the film was clean without being crystallized, “X” when the film was crystallized and the coated surface was very uneven, and “Δ” when the film was crystallized and its surface became cloudy after exposure and development.

TABLE 6

| | Residual film | Pattern | Chemical | | Thin film characteristics |
|---|---|---|---|---|---|
| Physical properties | ratio (%) | stability | resistance | Adhesiveness | (whitening) |
| Example 1 | 87.95 | ◯ | ◯ | ◯ | ◯ |
| Example 2 | 91.27 | ◯ | ◯ | ◯ | ◯ |
| Example 3 | 97.68 | ◯ | ◯ | ◯ | ◯ |
| Example 4 | 85.21 | ◯ | ◯ | ◯ | ◯ |
| Example 5 | 85.69 | ◯ | ◯ | ◯ | ◯ |
| Example 6 | 81.57 | ◯ | ◯ | ◯ | ◯ |
| Example 7 | 75.88 | ◯ | ◯ | ◯ | ◯ |
| Example 8 | 78.91 | ◯ | ◯ | ◯ | ◯ |
| Example 9 | 75.48 | ◯ | ◯ | ◯ | ◯ |
| Example 10 | 84.36 | ◯ | ◯ | ◯ | ◯ |
| Example 11 | 85.12 | ◯ | ◯ | ◯ | ◯ |
| Example 12 | 87.23 | ◯ | ◯ | ◯ | ◯ |
| Example 13 | 83.01 | ◯ | ◯ | ◯ | ◯ |
| Example 14 | 78.59 | ◯ | ◯ | ◯ | ◯ |
| Example 15 | 77.29 | ◯ | ◯ | ◯ | ◯ |
| Example 16 | 74.06 | ◯ | ◯ | ◯ | ◯ |
| Example 17 | 75.86 | ◯ | ◯ | ◯ | ◯ |
| Example 18 | 73.29 | ◯ | ◯ | ◯ | ◯ |
| Example 19 | 77.98 | ◯ | ◯ | ◯ | ◯ |
| Example 20 | 79.00 | ◯ | ◯ | ◯ | ◯ |
| Example 21 | 81.04 | ◯ | ◯ | ◯ | ◯ |
| Example 22 | 80.31 | ◯ | ◯ | ◯ | ◯ |
| Example 23 | 70.15 | ◯ | ◯ | ◯ | ◯ |
| Example 24 | 67.18 | ◯ | ◯ | ◯ | ◯ |
| Example 25 | 68.19 | ◯ | ◯ | ◯ | ◯ |
| Example 26 | 64.68 | ◯ | ◯ | ◯ | ◯ |
| Example 27 | 63.97 | ◯ | ◯ | ◯ | ◯ |
| Example 28 | 88.64 | ◯ | ◯ | ◯ | ◯ |
| Example 29 | 85.99 | ◯ | ◯ | ◯ | ◯ |
| Example 30 | 83.47 | ◯ | ◯ | ◯ | ◯ |
| Example 31 | 79.66 | ◯ | ◯ | ◯ | ◯ |
| Example 32 | 75.19 | ◯ | ◯ | ◯ | ◯ |
| Example 33 | 72.56 | ◯ | ◯ | ◯ | ◯ |
| Comparative Example 1 | 24.98 | X | X | X | ◯ |
| Comparative Example 2 | 72.97 | ◯ | ◯ | ◯ | X |
| Comparative Example 3 | 54.16 | ◯ | ◯ | ◯ | ◯ |
| Comparative Example 4 | 32.69 | X | X | ◯ | ◯ |

As can be seen from the results in Table 6 and FIG. 1, the inventive oxime ester phenylcarbazole compounds as photopolymerization initiators of photopolymerization compositions had high sensitivities even when used in small amounts, showed excellent physical properties in terms of residual film ratio, pattern stability, and chemical resistance. In addition, the thin films formed using the inventive oxime ester phenylcarbazole compounds did not undergo whitening. In conclusion, due to their excellent characteristics, the use of the inventive oxime ester phenylcarbazole compounds can minimize the occurrence of outgassing during exposure and post-baking for the production of resists for displays such as TFT-LCDs, OLEDs, and TSPs. This decreases the possibility of contamination, thus minimizing the number of defects.

1. An oxime ester phenylcarbazole compound as a photoinitiator for use in photocrosslinking wherein the carbon atom forming a double bond with the nitrogen atom in the oxime ester moiety is bonded to the phenylcarbazole group and is directly bonded to a (C₁-C₂₀)alkyl or (C₆-C₂₀)aryl group and wherein the phenylcarbazole group is substituted with one or more nitro groups.

2. The oxime ester phenylcarbazole compound according to claim 1, wherein the phenylcarbazole group is substituted with one or two nitro groups.

3. An oxime ester phenylcarbazole compound represented by Formula 1: 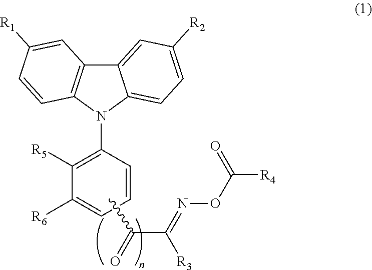
wherein R₁ and R₂ are each independently hydrogen, nitro, cyano, alkoxy or halogen, with the proviso that R₁ and R₂ are not simultaneously hydrogen, R₃ is (C₁-C₂₀)alkyl, (C₆-C₂₀)aryl, (C₁-C₂₀)alkoxy, (C₆-C₂₀)aryl(C₁-C₂₀)alkyl, hydroxy(C₁-C₂₀)alkyl, hydroxy(C₁-C₂₀)alkoxy(C₁-C₂₀)alkyl, (C₃-C₂₀)cycloalkyl, (C₁-C₂₀)alkylacyl or (C₆-C₂₀)arylacyl, R₄ is (C₁-C₂₀)alkyl, (C₆-C₂₀)aryl, (C₁-C₂₀)alkoxy, (C₆-C₂₀)aryl(C₁-C₂₀)alkyl, hydroxy(C₁-C₂₀)alkyl, hydroxy(C₁-C₂₀)alkoxy(C₁-C₂₀)alkyl, (C₃-C₂₀)cycloalkyl, (C₁-C₂₀)alkylacyl or (C₆-C₂₀)arylacyl; R₅ and R₆ are each independently hydrogen, (C₁-C₂₀)alkyl, (C₁-C₂₀)alkoxy, hydroxy(C₁-C₂₀)alkyl, hydroxy(C₁-C₂₀)alkoxy(C₁-C₂₀)alkyl, (C₃-C₂₀)cycloalkyl or (C₁-C₂₀)alkylacyl, and n is an integer of 0 or 1.

4. The oxime ester phenylcarbazole compound according to claim 3, wherein R₁ and R₂ in Formula 1 are each independently hydrogen, nitro, cyano, alkoxy or halogen, with the proviso that one of R₁ and R₂ is nitro.

5. The oxime ester phenylcarbazole compound according to claim 3, wherein R₃ in Formula 1 is (C₃-C₇)alkyl or (C₆-C₇)aryl.

6. The oxime ester phenylcarbazole compound according to claim 3, wherein R₄ in Formula 1 is (C₁-C₃)alkyl or (C₆-C₅)aryl.

7. The oxime ester phenylcarbazole compound according to claim 3, wherein R₅ and R₆ in Formula 1 are each independently hydrogen or (C₁-C₂)alkyl.

8. A photopolymerization initiator comprising the oxime ester phenylcarbazole compound according to claim 1.

9. A photopolymerizable composition comprising the oxime ester phenylcarbazole compound according to claim 1 and at least one compound selected from polymeric compounds soluble in a solvent or aqueous alkaline solution and photopolymerizable compounds having an ethylenically unsaturated bond.

10. A photopolymerizable composition comprising the oxime ester phenylcarbazole compound according to claim 1, at least one compound selected from polymeric compounds soluble in a solvent or an aqueous alkaline solution and photopolymerizable compounds having an ethylenically unsaturated bond, and a dye or pigment.

11. A black matrix formed from the photopolymerizable composition according to claim 9.

12. A color filter formed from the photopolymerizable composition according to claim 9.

13. A substrate having an organic insulating film formed from the photopolymerizable composition according to claim 9.

14. A substrate having a film formed by coating with the photopolymerizable composition according to claim 9.

15. The substrate according to claim 14, wherein the film is a display panel.
